(19) United States
(12) Patent Application Publication
(10) Pub. No.: US 2025/0161295 A1
(43) Pub. Date: May 22, 2025

(54) EXATECAN FORMULATION

(71) Applicants: Yale University, New Haven, CT (US); Stradefy Biosciences, Inc., New Haven, CT (US)

(72) Inventors: Michael Girardi, Madison, CT (US); W. Mark Saltzman, New Haven, CT (US); Julia Lewis, Clinton, CT (US); Heewon Suh, North Haven, CT (US); Brian Dixon, New Haven, CT (US)

(21) Appl. No.: 18/855,763

(22) PCT Filed: Apr. 17, 2023
(86) PCT No.: PCT/US2023/018831

§ 371 Date: Oct. 10, 2024

Related U.S. Application Data
(60) Provisional application No. 63/331,466, filed on Apr. 15, 2022.

(51) Int. Cl. A61K 31/4745; A61K 9/00; A61K 9/51; A61K 31/7125; A61P 35/00

(52) U.S. Cl. CPC A61K 31/4745; A61K 9/0019; A61K 9/5146; A61K 9/5192; A61K 31/7125; A61P 35/00

(57) ABSTRACT

Formulations for treating proliferative disorders have been developed. In some forms, the formulations are in the form of an injectable aqueous solution for treating tumors and contain exatecan mesylate, optionally including an immunostimulatory agent or adjuvant such as an immunostimulatory oligonucleotide (CpG), at a pH at which the exatecan is soluble until the pH is raised by contacting normal tissue. In some forms, the formulations contain core-shell particles, containing an exatecan in its free base form, a hydrophobic core, and a shell, coating, or corona containing hyperbranched polyglycerol in which hydroxyl groups of the hyperbranched polyglycerol are converted to aldehydes, to adhere the particles to tissue.

### CROSS-REFERENCE TO RELATED APPLICATIONS

This application claims benefit of and priority to U.S. Ser. No. 63/331,466 filed Apr. 15, 2022, which is incorporated herein by reference in its entirety.

### FIELD OF THE INVENTION

This invention is generally in the field of cancer treatment, particularly treatment of cutaneous cancers through intratumoral and/or peritumoral delivery of an exatecan or pharmaceutically acceptable salt thereof, alone or in combination with an immunostimulatory agent.

### BACKGROUND OF THE INVENTION

Keratinocyte-derived carcinomas, including squamous cell carcinoma (SCC), are among the most common malignancies. Surgical excision is the therapeutic standard but is not always clinically feasible, and currently available alternatives are limited to superficial tumors. Even with chemotherapy and/or radiation, outcomes are not always favorable.

In the United States, the frequency of cutaneous malignancies exceeds the frequency of all other cancers combined. Keratinocyte-derived carcinomas (KDCs), such as cutaneous SCC, make up the most common class of these malignancies, with SCCs responsible for approximately 5 million cases and 10,000 deaths annually in the United States. The incidence of cutaneous SCC has been steadily rising, with some estimates reporting greater than a 250% increase from 1976 to 1984 and 2000 to 2010. These numbers are projected to rise in proportion to the expanding elderly population, as well as the number of immunocompromised patients living with advanced disease, posing a significant public health challenge. Surgical excision is the most common first-line treatment for cutaneous SCC; however, SCC recurrence is common, with one study reporting rates as high as 50% of patients. Furthermore, surgical excision is undesirable in certain clinical settings, including at-risk patients with underlying bleeding diatheses and large-diameter tumors requiring complicated wound closures that increase the potential for postsurgical complications. The management and treatment of KDCs accounts for over $8.1 billion in US healthcare expenditures annually, a significant burden on both individuals and healthcare systems, necessitating efficient and effective alternatives.

Although both topical chemotherapeutic and immunomodulatory agents have demonstrated potential in the local treatment of superficial SCCs (Salim, Br. J. Dermatol. 148, 539-543 (2003); Morton Arch. Dermatol. 142, 729-735 (2006); Patel J. Am. Acad. Dermatol. 54, 1025-1032 (2006); Mackenzie-Wood, J. Am. Acad. Dermatol. 44, 462-470 (2001)), cream and gel formulations fail to achieve adequate penetration into deeper (e.g., nodular) SCCs. Moreover, topical chemotherapy (e.g., 5-fluorouracil) and immunotherapy (e.g., imiquimod) can diffuse from the site of application into the dense vasculature within tumors, with potential for diminished local efficacy and systemic toxicity (Kishi, Drug Saf. Case Rep. 5, 4 (2018).

Exatecan, a derivative of the chemotherapeutic, camptothecin, was designed to improve the safety, potency, and solubility of camptothecin in chemotherapy. While exatecan has shown potency and efficacy across a broad range of tumor models in vitro and in vivo, exatecan toxicity has hindered its further development as a systemically administered monotherapy, which include myelosuppression and life-threatening gastrointestinal toxicity. Accordingly, there remains a need for new formulations and methods for improved and safe local delivery of anti-cancer agents, such as exatecan.

Therefore, it is an object of the invention to provide formulations with improved safety and delivery for treating and/or reducing cancers.

### SUMMARY OF THE INVENTION

Formulations for treating or reducing proliferative disorders (such as cancers) have been developed for local or systemic delivery of a chemotherapeutic, preferably for the treatment or reduction of one or more forms of cutaneous cancer. In some forms, the formulation is administered by injecting into a tumor or tissue adjacent to or abutting the tumor to effectuate local delivery. In some forms, the formulation is administered intraperitoneally to a diseased tissue, such as that arising from peritoneal carcinomatosis that develops from metastasis of gastrointestinal and/or gynecological cancers.

In some forms, the formulation contains exatecan mesylate formulated in water, such as distilled water. In some forms, the formulation contains a combination of exatecan mesylate and an immunostimulatory agent, for example, an oligonucleotide such as CpG. Preferably, the pH of the formulation is maintained between 6.5 and 6.84 prior to administration.

In some forms, the formulation contains core-shell particles, containing an exatecan in its free base form, a hydrophobic core, and a shell, coating, or corona containing hyperbranched polyglycerol. The hydrophobic core contains a hydrophobic polymer covalently linked to a hyperbranched polyglycerol in the shell, coating, or corona. The exatecan is dispersed within the hydrophobic core. Further, some or all of the hydroxyl groups (e.g., vicinal hydroxyl groups) of the hyperbranched polyglycerol are converted to reactive functional groups, such as aldehydes, to adhere the particles to tissue. The data in the examples demonstrate enhanced retention at the site of delivery compared to exatecan administered in its unencapsulated form.

The formulations show effective killing of cancer cells such as cutaneous cancer cells and ovarian cancer cells. Further, the formulation shows low in vivo toxicity over a 14-day period or 56-day period, as determined by tracking the weights of subjects during this time frame. The formulation containing a combination of exatecan mesylate and an immnunostimulatory oligonucleotide (such as CpG) shows unexpectedly better efficacy in killing of cancer cells over a 49-day period.

Taken together, these results show that intratumoral/peritumoral delivery of a chemotherapeutic agent (e.g., an exatecan), with or without an immunostimulatory agent, represents a viable, non-surgical alternative for treating nodular cancers, such as cutaneous malignancy.

### BRIEF DESCRIPTION OF THE DRAWINGS

FIG. 1 is a scatter plot showing the histologic tumor area for mice harvested on day 16 after treatment with exatecan mesylate in distilled water.

FIG. 2 is a line graph showing the percent of mice with tumor less than 1 cm, treated with either distilled water or a 0.2 mg dose of exatecan mesylate, and observed over a 53-day period.

FIG. 3 is a survival curve showing the percent of mice that survived 100 days following a single injection of either UltraPure Distilled Water, Invitrogen, #10977-015 (dH₂O), control vehicle) or a water-based formulation containing 10 mg exatecan mesylate/mL. The total treatment volume was 0.02 mL. Mice were assessed to the endpoint at which the tumor volume was 1 cm³. Mantel-Cox log rank test was conducted 77 days post-treatment (p<0.0001).

FIG. 4 is a bar graph showing the dose dependent effect of exatecan treatment on tumor size in mice. Mice received a single intratumoral injection of either UltraPure Distilled Water, Invitrogen, #10977-015 (control vehicle) or one of the following concentrations of the water-based formulation containing exatecan mesylate: 10 mg/mL, 5 mg/mL, 2.5 mg/mL or 1.25 mg/mL for a total treatment volume of 0.02 mL. N=3 mice per group.

FIG. 5 is a scatter plot showing mice toxicity data (measured as percent weight loss) for treatment with vehicle (dH₂O), immunostimulatory agent (CpG at 0.5 mg/kg), free exatecan mesylate (Free EXA at 5 mg/kg), or Free EXA at a 5 mg/kg+CpG 0.5 mg/kg.

FIG. 6 is a survival curve showing the percent of mice that survived 49 days following a single injection of vehicle (dH₂O), immunostimulatory agent (CpG at 0.5 mg/kg), free exatecan mesylate (Free EXA at 5 mg/kg), or Free EXA at 5 mg/kg+CpG at 0.5 mg/kg.

FIGS. 7A and 7B are line graphs showing in vitro effect of exatecan encapsulated bioadhesive nanoparticles (BNP-111) on tumor cell viability: PDVC57 mouse squamous cell carcinoma (SCC) cells (FIG. 7A) or YUMMER1.7 mouse melanoma cells (FIG. 7B).

FIG. 8 is a bar graph showing BNP-111 tumor retention in PDVC57 SCC model. At the indicated time point(s), the bar on the left denotes free drug (if present), and the bar on the right denotes BNP-111. BNP-111 showed 37.77% drug retention at 72 hr.

FIGS. 9A and 9B are point graphs showing low dose BNP-111 in vivo efficacy in PDVC57 SCC model, as a function of percent weight loss (FIG. 9A) or histologic tumor area (FIG. 9B).

FIGS. 10A and 10B are a point graph (FIG. 10A) and a line graph (FIG. 10B) showing max dose BNP-111 in vivo efficacy in PDVC57 SCC model, as a function of percent weight loss (FIG. 10A) or percent survival (FIG. 10B).

FIGS. 11A-11C are a line graph (FIG. 11A) and point graphs (FIGS. 11B and 11C) showing BNP-111 in vivo efficacy in YUMMER1.7 melanoma model, as a function of tumor volume (FIG. 11A), tumor weight (FIG. 11B), or histologic tumor area (FIG. 11C).

FIG. 12 is a line graph showing BNP-111 in vivo efficacy in YUMMER1.7 melanoma model as a function of percent survival.

FIGS. 13A-13C are line graphs (FIGS. 13A and 13C) and an IVIS image (FIG. 13B) showing BNP-111 in vivo efficacy in ID8 ovarian cancer model.

FIGS. 14A and 14B are a line graph (FIG. 14A) and a bar graph (FIG. 14B) showing BNP-111 in vivo safety in ID8 ovarian cancer model, as a function of weight loss (FIG. 14B). In FIG. 14B the groups are bar graphs are, from left to right: Vehicle q7d x4; BNP-11, 1.25 mg/kg q7d x4; BNP-111 2.5 mg/kg q7d x4; Free SB-111 2.5 mg/kg q7d x4; and Free SB-111 10 mg/kg q7d x4.

FIGS. 15A and 15B are a line graph (FIG. 15A) and an IVIS image (FIG. 15B) showing retention of dye-conjugated BNPs IP post-injection.

### DETAILED DESCRIPTION OF THE INVENTION

### I. Definitions

“About” is intended to describe values either above or below the stated value in a range of approx. +/−10%. The ranges are intended to be made clear by context, and no further limitation is implied.

The phrase “pharmaceutically acceptable” refers to compositions, polymers and other materials and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Water soluble,” as relates to exatecan refers to a solubility of greater than 0.2 mg of exatecan dissolving in a mL of water, with or without warming. Suitable values for solubility include between 0.2 mg/mL of water and 10 mg/mL of water.

The term “pharmaceutically acceptable salts” refers to the modification of the original compound by making the acid or base salts thereof. Examples of pharmaceutically acceptable salts include, but are not limited to, mineral or organic acid salts of basic residues such as amines and alkali or organic salts of acidic residues such as carboxylic acids. For original compounds containing a basic residue, pharmaceutically acceptable salts can be prepared by treating the compounds with an appropriate amount of a non-toxic inorganic or organic acid. Suitable inorganic acids include hydrochloric, hydrobromic, sulfuric, sulfamic, phosphoric, and nitric acids; suitable organic acids include acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, naphthalenesulfonic, methanesulfonic, ethane disulfonic, oxalic, and isethionic acids. For original compounds containing an acidic residue, pharmaceutically acceptable salts can be prepared by treating the compounds with an appropriate amount of a non-toxic base.

Suitable non-toxic bases include ammonium hydroxide, sodium hydroxide, potassium hydroxide, lithium hydroxide, calcium hydroxide, magnesium hydroxide, ferrous hydroxide, zinc hydroxide, copper hydroxide, aluminum hydroxide, ferric hydroxide, isopropylamine, trimethylamine, diethylamine, triethylainine, tripropylanine, ethanolamine, 2-dimethylaminoethanol, 2-diethylaminoethanol, lysine, arginine, and histidine. Generally, pharmaceutically acceptable salts can be prepared by reacting the free acid or base form of the original compounds with a stoichiometric amount of the appropriate base or acid, respectively, in water or in an organic solvent, or in a mixture thereof. Non-aqueous media like ether, ethyl acetate, ethanol, isopropanol, acetonitrile, or combinations thereof can be used. Lists of suitable pharmaceutically acceptable salts can be found in Remington's Pharmaceutical Sciences, 20th Ed., Lippincott Williams & Wilkins, Baltimore, MD, 2000, p. 704; Handbook of Pharmaceutical Salts: Properties, Selection, and Use, Stahl and Wermuth, Eds., Wiley-VCH, Weinheim, 2002, and Kumar, et al., Pharmaceutical Technology-03-02-2008, Volume 32, Issue 3.

The term “treating” refers to treating or preventing a disease or disorder from occurring in an animal which may be predisposed to the disease or disorder, but has not yet been diagnosed as having it; inhibiting the disease or disorder, e.g., impeding its progress; and relieving the disease or disorder, e.g., causing regression of the disease or disorder. Treating the disease or disorder includes ameliorating at least one symptom of the particular disease or condition, even if the underlying pathophysiology is not affected, such as treating the cutaneous tumor of a subject by administration of an exatecan.

The term “therapeutically effective amount” refers to an amount of the therapeutic agent that, when incorporated into and/or onto particles described herein, produces a desired effect at a reasonable benefit/risk ratio, thereby alleviating one or more symptom of the disease or disorder. The effective amount may vary depending on such factors as the disease or disorder being treated, the size of the subject, or the severity of the disease or condition. In some embodiments, the term “effective amount” refers to an amount of a therapeutic agent or prophylactic agent to reduce or diminish one or more symptoms of a disease or disorder of a cancer, such as reducing tumor size (e.g., tumor volume).

“Substituted,” as used herein, refers to all permissible substituents of the compounds or functional groups described herein. In the broadest sense, the permissible substituents include acyclic and cyclic, branched and unbranched, carbocyclic and heterocyclic, aromatic and nonaromatic substituents of organic compounds. Illustrative substituents include, but are not limited to, halogens, hydroxyl groups, or any other organic groupings containing any number of carbon atoms, preferably 1-14 carbon atoms, and optionally include one or more heteroatoms such as oxygen, sulfur, or nitrogen grouping in linear, branched, or cyclic structural formats. Representative substituents include alkyl, substituted alkyl, alkenyl, substituted alkenyl, alkynyl, substituted alkynyl, phenyl, substituted phenyl, aryl, substituted aryl, heteroaryl, substituted heteroaryl, halo, hydroxyl, alkoxy, substituted alkoxy, phenoxy, substituted phenoxy, aroxy, substituted aroxy, alkylthio, substituted alkylthio, phenylthio, substituted phenylthio, arylthio, substituted arylthio, cyano, isocyano, substituted isocyano, carbonyl, substituted carbonyl, carboxyl, substituted carboxyl, amino, substituted amino, amido, substituted amido, sulfonyl, substituted sulfonyl, sulfonic acid, phosphoryl, substituted phosphoryl, phosphonyl, substituted phosphonyl, polyaryl, substituted polyaryl, C₃-C₂₀ cyclic, substituted C₃-C₂₀ cyclic, C₁-C₂₀ heterocyclic, substituted C₁-C₂₀ heterocyclic, amino acid, poly(lactic-co-glycolic acid), peptide, and polypeptide groups. Such alkyl, substituted alkyl, alkenyl, substituted alkenyl, alkynyl, substituted alkynyl, phenyl, substituted phenyl, aryl, substituted aryl, heteroaryl, substituted heteroaryl, halo, hydroxyl, alkoxy, substituted alkoxy, phenoxy, substituted phenoxy, aroxy, substituted aroxy, alkylthio, substituted alkylthio, phenylthio, substituted phenylthio, arylthio, substituted arylthio, cyano, isocyano, substituted isocyano, carbonyl, substituted carbonyl, carboxyl, substituted carboxyl, amino, substituted amino, amido, substituted amido, sulfonyl, substituted sulfonyl, sulfonic acid, phosphoryl, substituted phosphoryl, phosphonyl, substituted phosphonyl, polyaryl, substituted polyaryl, C₃-C₂₀ cyclic, substituted C₃-C₂₀ cyclic, heterocyclic, substituted heterocyclic, amino acid, poly(lactic-co-glycolic acid), poly(lactic-co-glycolic acid), peptide, and polypeptide groups can be further substituted.

Heteroatoms such as nitrogen may have hydrogen substituents and/or any permissible substituents of organic compounds described herein which satisfy the valences of the heteroatoms. It is understood that “substitution” or “substituted” includes the implicit proviso that such substitution is in accordance with permitted valence of the substituted atom and the substituent, and that the substitution results in a stable compound, i.e. a compound that does not spontaneously undergo transformation such as by rearrangement, cyclization, elimination, etc.

Except where specifically and expressly provided to the contrary, the term “substituted” refers to a structure, e.g., a chemical compound or a moiety on a larger chemical compound, regardless of how the structure was formed. The structure is not limited to a structure made by any specific method.

“Aryl,” as used herein, refers to C₅-C₂₆-membered aromatic or fused aromatic ring systems. Examples of aromatic groups are benzene, naphthalene, anthracene, phenanthrene, chrysene, pyrene, corannulene, coronene, etc.

The term “substituted aryl” refers to an aryl group, wherein one or more hydrogen atoms on one or more aromatic rings are substituted with one or more substituents including, but not limited to, halogen, azide, alkyl, aralkyl, alkenyl, alkynyl, cycloalkyl, hydroxyl, alkoxy, carbonyl (such as a ketone, aldehyde, carboxyl, alkoxycarbonyl, formyl, or an acyl), silyl (such as trimethylsilyl), ether, ester, thiocarbonyl (such as a thioester, a thioacetate, or a thioformate), alkoxyl, phosphoryl, phosphate, phosphonate, phosphinate, amino (or quarternized amino), amido, amidine, imine, cyano, nitro, azido, sulfhydryl, imino, alkylthio, sulfate, sulfonate, sulfamoyl, sulfoxide, sulfonamido, sulfonyl, heterocyclyl, alkylaryl, haloalkyl (such as CF₃, —CH₂—CF₃, —CCl₃), —CN, aryl, heteroaryl, and combinations thereof.

“Heterocycle,” “heterocyclic” and “heterocyclyl” are used interchangeably, and refer to a cyclic radical attached via a ring carbon or nitrogen atom of a monocyclic or bicyclic ring containing 3-10 ring atoms, and preferably from 5-6 ring atoms, consisting of carbon and one to four heteroatoms each selected from the group consisting of non-peroxide oxygen, sulfur, and N(Y) wherein Y is absent or is H, O, C₁-C₁₀ alkyl, phenyl or benzyl, and optionally containing 1-3 double bonds and optionally substituted with one or more substituents. Heterocyclyl are distinguished from heteroaryl by definition. Examples of heterocycles include, but are not limited to piperazinyl, piperidinyl, piperidonyl, 4-piperidonyl, dihydrofuro[2,3-b]tetrahydrofuran, morpholinyl, piperazinyl, piperidinyl, piperidonyl, 4-piperidonyl, piperonyl, pyranyl, 2H-pyrrolyl, 4H-quinolizinyl, quinuclidinyl, tetrahydrofuranyl, 6H-1,2,5-thiadiazinyl. Heterocyclic groups can optionally be substituted with one or more substituents as defined above for alkyl and aryl.

The term “heteroaryl” refers to C₅-C₂₆-membered aromatic or fused aromatic ring systems, in which one or more carbon atoms on one or more aromatic ring structures have been substituted with a heteroatom. Suitable heteroatoms include, but are not limited to, oxygen, sulfur, and nitrogen. Examples of heteroaryl groups pyrrole, furan, thiophene, imidazole, oxazole, thiazole, triazole, tetrazole, pyrazole, pyridine, pyrazine, pyridazine and pyrimidine, and the like. Examples of heteroaryl rings include, but are not limited to, benzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzimidazolinyl, carbazolyl, 4aH-carbazolyl, carbolinyl, chromanyl, chromenyl, cinnolinyl, decahydroquinolinyl, 2H,6H-1,5,2-dithiazinyl, furanyl, furazanyl, imidazolidinyl, imidazolinyl, imidazolyl, 1H-indazolyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isoxazolyl, methylenedioxyphenyl, naphthyridinyl, octahydroisoquinolinyl, 1,2,3-oxadiazolyl, 1,2,4-oxadiazolyl, 1,2,5-oxadiazolyl, 1,3,4-oxadiazolyl, oxazolidinyl, oxazolyl, oxindolyl, pyrimidinyl, phenanthridinyl, phenanthrolinyl, phenazinyl, phenothiazinyl, phenoxathinyl, phenoxazinyl, phthalazinyl, pteridinyl, purinyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, pyrrolyl, quinazolinyl, quinolinyl, quinoxalinyl, tetrahydroisoquinolinyl, tetrahydroquinolinyl, tetrazolyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl and xanthenyl. One or more of the rings can be substituted as defined below for “substituted heteroaryl.”

The term “substituted heteroaryl” refers to a heteroaryl group in which one or more hydrogen atoms on one or more heteroaromatic rings are substituted with one or more substituents including, but not limited to, halogen, azide, alkyl, aralkyl, alkenyl, alkynyl, cycloalkyl, hydroxyl, alkoxy, carbonyl (such as a ketone, aldehyde, carboxyl, alkoxycarbonyl, formyl, or an acyl), silyl (such as trimethylsilyl), ether, ester, thiocarbonyl (such as a thioester, a thioacetate, or a thioformate), alkoxyl, phosphoryl, phosphate, phosphonate, phosphinate, amino (or quarternized amino), amido, amidine, imine, cyano, nitro, azido, sulfhydryl, imino, alkylthio, sulfate, sulfonate, sulfamoyl, sulfoxide, sulfonamido, sulfonyl, heterocyclyl, alkylaryl, haloalkyl (such as CF₃, —CH₂—CF₃, —CCl₃), —CN, aryl, heteroaryl, and combinations thereof.

“Alkyl,” as used herein, refers to the radical of saturated aliphatic groups, including straight-chain alkyl groups, branched-chain alkyl, cycloalkyl (alicyclic), alkyl substituted cycloalkyl groups, and cycloalkyl substituted alkyl. In preferred embodiments, a straight chain or branched chain alkyl has 30 or fewer carbon atoms in its backbone (e.g., C₁-C₃₀ for straight chains, C₃-C₃₀ for branched chains), preferably 20 or fewer, more preferably 15 or fewer, most preferably 10 or fewer. Likewise, preferred cycloalkyls have from 3-10 carbon atoms in their ring structure, and more preferably have 5, 6 or 7 carbons in the ring structure. The term “alkyl” (or “lower alkyl”) as used throughout the specification, examples, and claims is intended to include both “unsubstituted alkyls” and “substituted alkyls,” the latter of which refers to alkyl moieties having one or more substituents replacing a hydrogen on one or more carbons of the hydrocarbon backbone. Such substituents include, but are not limited to, halogen, hydroxyl, carbonyl (such as a carboxyl, alkoxycarbonyl, formyl, or an acyl), thiocarbonyl (such as a thioester, a thioacetate, or a thioformate), alkoxyl, phosphoryl, phosphate, phosphonate, phosphinate, amino, amido, amidine, imine, cyano, nitro, azido, sulfhydryl, alkylthio, sulfate, sulfonate, sulfamoyl, sulfoxide, sulfonamido, sulfonyl, heterocyclyl, aralkyl, or an aromatic or heteroaromatic moiety.

Unless the number of carbons is otherwise specified, “lower alkyl” as used herein means an alkyl group, as defined above, but having from one to ten carbons, more preferably from one to six carbon atoms in its backbone structure. Likewise, “lower alkenyl” and “lower alkynyl” have similar chain lengths. Throughout the application, preferred alkyl groups are lower alkyls. In preferred embodiments, a substituent designated herein as alkyl is a lower alkyl.

“Alkyl” includes one or more substitutions at one or more carbon atoms of the hydrocarbon radical as well as heteroalkyls. Suitable substituents include, but are not limited to, halogens, such as fluorine, chlorine, bromine, or iodine; hydroxyl; —NRR′, wherein R and R′ are independently hydrogen, alkyl, or aryl, and wherein the nitrogen atom is optionally quaternized; —SR, wherein R is hydrogen, alkyl, or aryl; —CN; —NO2; —COOH; carboxylate; —COR, —COOR, or —CON(R)₂, wherein R is hydrogen, alkyl, or aryl; azide, aralkyl, alkoxyl, imino, phosphonate, phosphinate, silyl (such as trimethylsilyl), ether, sulfonyl, sulfonamido, heterocyclyl, aromatic or heteroaromatic moieties, haloalkyl (such as —CF₃, —CH₂—CF₃, —CCl₃); —CN; —NCOCOCH₂CH₂; —NCOCOCHCH; —NCS; and combinations thereof.

It will be understood by those skilled in the art that the moieties substituted on the hydrocarbon chain can themselves be substituted, if appropriate. For instance, the substituents of a substituted alkyl may include halogen, hydroxy, nitro, thiols, amino, azido, imino, amido, phosphoryl (including phosphonate and phosphinate), sulfonyl (including sulfate, sulfonamido, sulfamoyl, sulfoxide and sulfonate), and silyl (such as trimethylsilyl) groups, as well as ethers, alkylthios, carbonyls (including ketones, aldehydes, carboxylates, and esters), haloalkyls, —CN and the like. Cycloalkyls can be substituted in the same manner.

The terms “alkenyl” and “alkynyl” refer to unsaturated aliphatic groups analogous in length and possible substitution to the alkyls described above, but that contain at least one double or triple bond respectively.

The term “substituted alkenyl” refers to alkenyl moieties having one or more substituents replacing one or more hydrogen atoms on one or more carbons of the hydrocarbon backbone. Such substituents include, but are not limited to, halogen, azide, alkyl, aralkyl, alkenyl, alkynyl, cycloalkyl, hydroxyl, carbonyl (such as a carboxyl, alkoxycarbonyl, formyl, or an acyl), silyl (such as trimethylsilyl), ether, ester, thiocarbonyl (such as a thioester, a thioacetate, or a thioformate), alkoxyl, phosphoryl, phosphate, phosphonate, phosphinate, amino (or quarternized amino), amido, amidine, imine, cyano, nitro, azido, sulfhydryl, alkylthio, sulfate, sulfonate, sulfamoyl, sulfoxide, sulfonamido, sulfonyl, heterocyclyl, alkylaryl, haloalkyl, —CN, aryl, heteroaryl, and combinations thereof.

The term “substituted alkynyl” refers to alkynyl moieties having one or more substituents replacing one or more hydrogen atoms on one or more carbons of the hydrocarbon backbone. Such substituents include, but are not limited to, halogen, azide, alkyl, aralkyl, alkenyl, alkynyl, cycloalkyl, hydroxyl, carbonyl (such as a carboxyl, alkoxycarbonyl, formyl, or an acyl), silyl (such as trimethylsilyl), ether, ester, thiocarbonyl (such as a thioester, a thioacetate, or a thioformate), alkoxyl, phosphoryl, phosphate, phosphonate, phosphinate, amino (or quarternized amino), amido, amidine, imine, cyano, nitro, azido, sulfhydryl, alkylthio, sulfate, sulfonate, sulfamoyl, sulfoxide, sulfonamido, sulfonyl, heterocyclyl, alkylaryl, haloalkyl, —CN, aryl, heteroaryl, and combinations thereof.

The term “phenyl” is art recognized, and refers to the aromatic moiety —C₆H₅, i.e., a benzene ring without one hydrogen atom.

The term “substituted phenyl” refers to a phenyl group, as defined above, having one or more substituents replacing one or more hydrogen atoms on one or more carbons of the phenyl ring. Such substituents include, but are not limited to, halogen, azide, alkyl, aralkyl, alkenyl, alkynyl, cycloalkyl, hydroxyl, carbonyl (such as a carboxyl, alkoxycarbonyl, formyl, or an acyl), silyl (such as trimethylsilyl), ether, ester, thiocarbonyl (such as a thioester, a thioacetate, or a thioformate), alkoxyl, phosphoryl, phosphate, phosphonate, phosphinate, amino (or quarternized amino), amido, amidine, imine, cyano, nitro, azido, sulfhydryl, alkylthio, sulfate, sulfonate, sulfamoyl, sulfoxide, sulfonamido, sulfonyl, heterocyclyl, alkylaryl, haloalkyl, —CN, aryl, heteroaryl, and combinations thereof.

“Amino” and “Amine,” as used herein, are art-recognized and refer to both substituted and unsubstituted amines, e.g., a moiety that can be represented by the general formula:

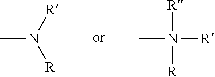

wherein, R, R′, and R″ each independently represent a hydrogen, substituted or unsubstituted alkyl, substituted or unsubstituted alkenyl, substituted or unsubstituted alkynyl, substituted or unsubstituted carbonyl, —(CH₂)ₘ—R″′, or R and R′ taken together with the N atom to which they are attached complete a heterocycle having from 3 to 14 atoms in the ring structure; R″′ represents a hydroxy group, substituted or unsubstituted carbonyl group, an aryl, a cycloalkyl ring, a cycloalkenyl ring, a heterocycle, or a polycycle; and m is zero or an integer ranging from 1 to 8. In preferred embodiments, only one of R and R′ can be a carbonyl, e.g., R and R′ together with the nitrogen do not form an imide. In preferred embodiments, R and R′ (and optionally R″) each independently represent a hydrogen atom, substituted or unsubstituted alkyl, a substituted or unsubstituted alkenyl, or —(CH₂)ₘ—R″′. Thus, the term ‘alkylamine’ as used herein refers to an amine group, as defined above, having a substituted or unsubstituted alkyl attached thereto (i.e. at least one of R, R′, or R″ is an alkyl group).

“Carbonyl,” as used herein, is art-recognized and includes such moieties as can be represented by the general formula:

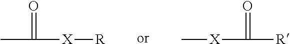

wherein X is a bond, or represents an oxygen or a sulfur, and R represents a hydrogen, a substituted or unsubstituted alkyl, substituted or unsubstituted alkenyl, substituted or unsubstituted alkynyl, substituted or unsubstituted cycloalkyl, substituted or unsubstituted heterocyclyl, substituted or unsubstituted alkylaryl, substituted or unsubstituted arylalkyl, substituted or unsubstituted aryl, or substituted or unsubstituted heteroaryl, —(CH₂)ₘ—R″, or a pharmaceutical acceptable salt, R′ represents a hydrogen, substituted or unsubstituted alkyl, substituted or unsubstituted alkenyl, substituted or unsubstituted alkynyl, substituted or unsubstituted cycloalkyl, substituted or unsubstituted heterocyclyl, substituted or unsubstituted alkylaryl, substituted or unsubstituted arylalkyl, substituted or unsubstituted aryl, or substituted or unsubstituted heteroaryl or —(CH₂)ₘ—R″; R″ represents a hydroxy group, substituted or unsubstituted carbonyl group, an aryl, a cycloalkyl ring, a cycloalkenyl ring, a heterocycle, or a polycycle; and m is zero or an integer ranging from 1 to 8. Where X is oxygen and R is defines as above, the moiety is also referred to as a carboxyl group. When X is oxygen and R is hydrogen, the formula represents a ‘carboxylic acid.’ Where X is oxygen and R′ is hydrogen, the formula represents a ‘formate.’ Where X is oxygen and R or R′ is not hydrogen, the formula represents an “ester”. In general, where the oxygen atom of the above formula is replaced by a sulfur atom, the formula represents a ‘thiocarbonyl’ group. Where X is sulfur and R or R′ is not hydrogen, the formula represents a ‘thioester.’ Where X is sulfur and R is hydrogen, the formula represents a ‘thiocarboxylic acid.’ Where X is sulfur and R′ is hydrogen, the formula represents a ‘thioformate.’ Where X is a bond and R is not hydrogen, the above formula represents a ‘ketone.’ Where X is a bond and R is hydrogen, the above formula represents an ‘aldehyde.’

The term “substituted carbonyl” refers to a carbonyl, as defined above, wherein one or more hydrogen atoms in R, R′ or a group to which the moiety

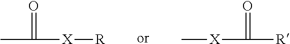

is attached, are independently substituted. Such substituents include, but are not limited to, halogen, azide, alkyl, aralkyl, alkenyl, alkynyl, cycloalkyl, hydroxyl, carbonyl (such as a carboxyl, alkoxycarbonyl, formyl, or an acyl), silyl (such as trimethylsilyl), ether, ester, thiocarbonyl (such as a thioester, a thioacetate, or a thioformate), alkoxyl, phosphoryl, phosphate, phosphonate, phosphinate, amino (or quarternized amino), amido, amidine, imine, cyano, nitro, azido, sulfhydryl, alkylthio, sulfate, sulfonate, sulfamoyl, sulfoxide, sulfonamido, sulfonyl, heterocyclyl, alkylaryl, haloalkyl, —CN, aryl, heteroaryl, and combinations thereof.

The term “carboxyl” is as defined above for the formula

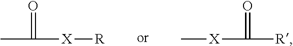

and is defined more specifically by the formula —R^{iv} COOH, wherein R^{iv} is an alkyl, alkenyl, alkynyl, cycloalkyl, heterocyclyl, alkylaryl, arylalkyl, aryl, or heteroaryl. In preferred embodiments, a straight chain or branched chain alkyl, alkenyl, and alkynyl have 30 or fewer carbon atoms in its backbone (e.g., C₁-C₃₀ for straight chain alkyl, C₃-C₃₀ for branched chain alkyl, C₂-C₃₀ for straight chain alkenyl and alkynyl, C₃-C₃₀ for branched chain alkenyl and alkynyl), preferably 20 or fewer, more preferably 15 or fewer, most preferably 10 or fewer. Likewise, preferred cycloalkyls, heterocyclyls, aryls and heteroaryls have from 3-10 carbon atoms in their ring structure, and more preferably have 5, 6 or 7 carbons in the ring structure.

The term “substituted carboxyl” refers to a carboxyl, as defined above, wherein one or more hydrogen atoms in R^{iv} are substituted. Such substituents include, but are not limited to, halogen, azide, alkyl, aralkyl, alkenyl, alkynyl, cycloalkyl, hydroxyl, carbonyl (such as a carboxyl, alkoxycarbonyl, formyl, or an acyl), silyl (such as trimethylsilyl), ether, ester, thiocarbonyl (such as a thioester, a thioacetate, or a thioformate), alkoxyl, phosphoryl, phosphate, phosphonate, phosphinate, amino (or quarternized amino), amido, amidine, imine, cyano, nitro, azido, sulfhydryl, alkylthio, sulfate, sulfonate, sulfamoyl, sulfoxide, sulfonamido, sulfonyl, heterocyclyl, alkylaryl, haloalkyl, —CN, aryl, heteroaryl, and combinations thereof.

“Heteroalkyl,” as used herein, refers to straight or branched chain, or cyclic carbon-containing radicals, or combinations thereof, containing at least one heteroatom. Suitable heteroatoms include, but are not limited to, O, N, Si, P and S, wherein the nitrogen, phosphorous and sulfur atoms are optionally oxidized, and the nitrogen heteroatom is optionally quaternized.

Examples of saturated hydrocarbon radicals include, but are not limited to, methyl, ethyl, n-propyl, isopropyl, n-butyl, t-butyl, isobutyl, sec-butyl, cyclohexyl, (cyclohexyl)methyl, cyclopropylmethyl, and homologs and isomers of, for example, n-pentyl, n-hexyl, n-heptyl, n-octyl. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, and 3-butynyl.

The terms “alkoxyl” or “alkoxy,” “aroxy” or “aryloxy,” generally describe compounds represented by the formula —OR^{v}, wherein R^{V} includes, but is not limited to, substituted or unsubstituted alkyl, alkenyl, alkynyl, cycloalkyl, heterocyclyl, cycloalkenyl, heterocycloalkenyl, aryl, heteroaryl, arylalkyl, heteroalkyls, alkylaryl, alkylheteroaryl.

The terms “alkoxyl” or “alkoxy” as used herein refer to an alkyl group, as defined above, having an oxygen radical attached thereto. Representative alkoxyl groups include methoxy, ethoxy, propyloxy, tert-butoxy and the like. An “ether” is two hydrocarbons covalently linked by an oxygen. Accordingly, the substituent of an alkyl that renders that alkyl an ether is or resembles an alkoxyl, such as can be represented by one of —O— alkyl, —O-alkenyl, and —O-alkynyl. The term alkoxy also includes cycloalkyl, heterocyclyl, cycloalkenyl, heterocycloalkenyl, and arylalkyl having an oxygen radical attached to at least one of the carbon atoms, as valency permits.

The term “substituted alkoxy” refers to an alkoxy group having one or more substituents replacing one or more hydrogen atoms on one or more carbons of the alkoxy backbone. Such substituents include, but are not limited to, halogen, azide, alkyl, aralkyl, alkenyl, alkynyl, cycloalkyl, hydroxyl, carbonyl (such as a carboxyl, alkoxycarbonyl, formyl, or an acyl), silyl (such as trimethylsilyl), ether, ester, thiocarbonyl (such as a thioester, a thioacetate, or a thioformate), alkoxyl, phosphoryl, phosphate, phosphonate, phosphinate, amino (or quarternized amino), amido, amidine, imine, cyano, nitro, azido, sulfhydryl, alkylthio, sulfate, sulfonate, sulfamoyl, sulfoxide, sulfonamido, sulfonyl, heterocyclyl, alkylaryl, haloalkyl, —CN, aryl, heteroaryl, and combinations thereof.

The term “phenoxy” is art recognized, and refers to a compound of the formula —OR^{v} wherein R^{V} is (i.e., —O—C₆H₅). One of skill in the art recognizes that a phenoxy is a species of the aroxy genus.

The term “substituted phenoxy” refers to a phenoxy group, as defined above, having one or more substituents replacing one or more hydrogen atoms on one or more carbons of the phenyl ring. Such substituents include, but are not limited to, halogen, azide, alkyl, aralkyl, alkenyl, alkynyl, cycloalkyl, hydroxyl, carbonyl (such as a carboxyl, alkoxycarbonyl, formyl, or an acyl), silyl (such as trimethylsilyl), ether, ester, thiocarbonyl (such as a thioester, a thioacetate, or a thioformate), alkoxyl, phosphoryl, phosphate, phosphonate, phosphinate, amino (or quarternized amino), amido, amidine, imine, cyano, nitro, azido, sulfhydryl, alkylthio, sulfate, sulfonate, sulfamoyl, sulfoxide, sulfonamido, sulfonyl, heterocyclyl, alkylaryl, haloalkyl, —CN, aryl, heteroaryl, and combinations thereof.

The terms “aroxy” and “aryloxy,” as used interchangeably herein, are represented by —O-aryl or —O-heteroaryl, wherein aryl and heteroaryl are as defined herein.

The terms “substituted aroxy” and “substituted aryloxy,” as used interchangeably herein, represent —O-aryl or —O-heteroaryl, having one or more substituents replacing one or more hydrogen atoms on one or more ring atoms of the aryl and heteroaryl, as defined herein. Such substituents include, but are not limited to, halogen, azide, alkyl, aralkyl, alkenyl, alkynyl, cycloalkyl, hydroxyl, carbonyl (such as a carboxyl, alkoxycarbonyl, formyl, or an acyl), silyl (such as trimethylsilyl), ether, ester, thiocarbonyl (such as a thioester, a thioacetate, or a thioformate), alkoxyl, phosphoryl, phosphate, phosphonate, phosphinate, amino (or quarternized amino), amido, amidine, imine, cyano, nitro, azido, sulfhydryl, alkylthio, sulfate, sulfonate, sulfamoyl, sulfoxide, sulfonamido, sulfonyl, heterocyclyl, alkylaryl, haloalkyl, —CN, aryl, heteroaryl, and combinations thereof.

The term “alkylthio” refers to an alkyl group, as defined above, having a sulfur radical attached thereto. The “alkylthio” moiety is represented by —S-alkyl. Representative alkylthio groups include methylthio, ethylthio, and the like. The term “alkylthio” also encompasses cycloalkyl groups having a sulfur radical attached thereto.

The term “substituted alkylthio” refers to an alkylthio group having one or more substituents replacing one or more hydrogen atoms on one or more carbon atoms of the alkylthio backbone. Such substituents include, but are not limited to, halogen, azide, alkyl, aralkyl, alkenyl, alkynyl, cycloalkyl, hydroxyl, carbonyl (such as a carboxyl, alkoxycarbonyl, formyl, or an acyl), silyl (such as trimethylsilyl), ether, ester, thiocarbonyl (such as a thioester, a thioacetate, or a thioformate), alkoxyl, phosphoryl, phosphate, phosphonate, phosphinate, amino (or quarternized amino), amido, amidine, imine, cyano, nitro, azido, sulfhydryl, alkylthio, sulfate, sulfonate, sulfamoyl, sulfoxide, sulfonamido, sulfonyl, heterocyclyl, alkylaryl, haloalkyl, —CN, aryl, heteroaryl, and combinations thereof.

The term “phenylthio” is art recognized, and refers to —S—C₆H₅, i.e., a phenyl group attached to a sulfur atom.

The term “substituted phenylthio” refers to a phenylthio group, as defined above, having one or more substituents replacing a hydrogen on one or more carbons of the phenyl ring. Such substituents include, but are not limited to, halogen, azide, alkyl, aralkyl, alkenyl, alkynyl, cycloalkyl, hydroxyl, carbonyl (such as a carboxyl, alkoxycarbonyl, formyl, or an acyl), silyl (such as trimethylsilyl), ether, ester, thiocarbonyl (such as a thioester, a thioacetate, or a thioformate), alkoxyl, phosphoryl, phosphate, phosphonate, phosphinate, amino (or quarternized amino), amido, amidine, imine, cyano, nitro, azido, sulfhydryl, alkylthio, sulfate, sulfonate, sulfamoyl, sulfoxide, sulfonamido, sulfonyl, heterocyclyl, alkylaryl, haloalkyl, —CN, aryl, heteroaryl, and combinations thereof.

“Arylthio” refers to —S-aryl or —S-heteroaryl groups, wherein aryl and heteroaryl as defined herein.

The term “substituted arylthio” represents —S-aryl or —S-heteroaryl, having one or more substituents replacing a hydrogen atom on one or more ring atoms of the aryl and heteroaryl rings as defined herein. Such substituents include, but are not limited to, halogen, azide, alkyl, aralkyl, alkenyl, alkynyl, cycloalkyl, hydroxyl, carbonyl (such as a carboxyl, alkoxycarbonyl, formyl, or an acyl), silyl (such as trimethylsilyl), ether, ester, thiocarbonyl (such as a thioester, a thioacetate, or a thioformate), alkoxyl, phosphoryl, phosphate, phosphonate, phosphinate, amino (or quarternized amino), amido, amidine, imine, cyano, nitro, azido, sulfhydryl, alkylthio, sulfate, sulfonate, sulfamoyl, sulfoxide, sulfonamido, sulfonyl, heterocyclyl, alkylaryl, haloalkyl, —CN, aryl, heteroaryl, and combinations thereof.

“Arylalkyl,” as used herein, refers to an alkyl group that is substituted with a substituted or unsubstituted aryl group. When a heteroaryl group is involved, the chemical moiety can be referred to as a “heteroarylalkyl.”

“Alkylaryl,” as used herein, refers to an aryl group that is substituted with a substituted or unsubstituted alkyl group. When a heteroaryl group is involved, the chemical moiety can be referred to as a “alkylheteroaryl.”

The terms “amide” or “amido” are used interchangeably, refer to both “unsubstituted amido” and “substituted amido” and are represented by the general formula:

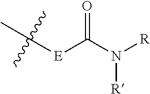

wherein, E is absent, or E is substituted or unsubstituted alkyl, substituted or unsubstituted alkenyl, substituted or unsubstituted alkynyl, substituted or unsubstituted aralkyl, substituted or unsubstituted cycloalkyl, substituted or unsubstituted aryl, substituted or unsubstituted heteroaryl, substituted or unsubstituted heterocyclyl, wherein independently of E, R and R′ each independently represent a hydrogen, substituted or unsubstituted alkyl, substituted or unsubstituted alkenyl, substituted or unsubstituted alkynyl, substituted or unsubstituted carbonyl, substituted or unsubstituted cycloalkyl, substituted or unsubstituted heterocyclyl, substituted or unsubstituted alkylaryl, substituted or unsubstituted arylalkyl, substituted or unsubstituted aryl, or substituted or unsubstituted heteroaryl, —(CH₂)ₘ—R″′, or R and R′ taken together with the N atom to which they are attached complete a heterocycle having from 3 to 14 atoms in the ring structure; R″′ represents a hydroxy group, substituted or unsubstituted carbonyl group, an aryl, a cycloalkyl ring, a cycloalkenyl ring, a heterocycle, or a polycycle; and m is zero or an integer ranging from 1 to 8. In preferred embodiments, only one of R and R′ can be a carbonyl, e.g., R and R′ together with the nitrogen do not form an imide. In preferred embodiments, R and R′ each independently represent a hydrogen atom, substituted or unsubstituted alkyl, a substituted or unsubstituted alkenyl, or —(CH2)ₘ—R″′. When E is oxygen, a carbamate is formed. The carbamate cannot be attached to another chemical species, such as to form an oxygen-oxygen bond, or other unstable bonds, as understood by one of ordinary skill in the art.

The term “sulfonyl” is represented by the formula

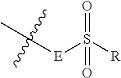

wherein E is absent, or E is alkyl, alkenyl, alkynyl, aralkyl, alkylaryl, cycloalkyl, aryl, heteroaryl, heterocyclyl, wherein independently of E, R represents a hydrogen, substituted or unsubstituted alkyl, substituted or unsubstituted alkenyl, substituted or unsubstituted alkynyl, substituted or unsubstituted amine, substituted or unsubstituted cycloalkyl, substituted or unsubstituted heterocyclyl, substituted or unsubstituted alkylaryl, substituted or unsubstituted arylalkyl, substituted or unsubstituted aryl, or substituted or unsubstituted heteroaryl, —(CH₂)ₘ—R′″, or E and R taken together with the S atom to which they are attached complete a heterocycle having from 3 to 14 atoms in the ring structure; R″′ represents a hydroxy group, substituted or unsubstituted carbonyl group, an aryl, a cycloalkyl ring, a cycloalkenyl ring, a heterocycle, or a polycycle; and m is zero or an integer ranging from 1 to 8. In preferred embodiments, only one of E and R can be substituted or unsubstituted amine, to form a “sulfonamide” or “sulfonamido.” The substituted or unsubstituted amine is as defined above.

The term “substituted sulfonyl” represents a sulfonyl in which E and R are independently substituted. Such substituents include, but are not limited to, halogen, azide, alkyl, aralkyl, alkenyl, alkynyl, cycloalkyl, hydroxyl, carbonyl (such as a carboxyl, alkoxycarbonyl, formyl, or an acyl), silyl (such as trimethylsilyl), ether, ester, thiocarbonyl (such as a thioester, a thioacetate, or a thioformate), alkoxyl, phosphoryl, phosphate, phosphonate, phosphinate, amino (or quarternized amino), amido, amidine, imine, cyano, nitro, azido, sulfhydryl, alkylthio, sulfate, sulfonate, sulfamoyl, sulfoxide, sulfonamido, sulfonyl, heterocyclyl, alkylaryl, haloalkyl, —CN, aryl, heteroaryl, and combinations thereof.

The term “sulfonic acid” refers to a sulfonyl, as defined above, wherein R is hydroxyl, and E is absent, or E is substituted or unsubstituted cycloalkyl, substituted or unsubstituted heterocyclyl, substituted or unsubstituted alkylaryl, substituted or unsubstituted arylalkyl, substituted or unsubstituted aryl, or substituted or unsubstituted heteroaryl.

The term “sulfate” refers to a sulfonyl, as defined above, wherein E is absent, oxygen, alkoxy, aroxy, substituted alkoxy or substituted aroxy, as defined above, and R is independently hydroxyl, alkoxy, aroxy, substituted alkoxy or substituted aroxy, as defined above. When E is oxygen, the sulfate cannot be attached to another chemical species, such as to form an oxygen-oxygen bond, or other unstable bonds, as understood by one of ordinary skill in the art.

The term “sulfonate” refers to a sulfonyl, as defined above, wherein E is oxygen, alkoxy, aroxy, substituted alkoxy or substituted aroxy, as defined above, and R is independently hydrogen, substituted or unsubstituted alkyl, substituted or unsubstituted alkenyl, substituted or unsubstituted alkynyl, substituted or unsubstituted amine, substituted or unsubstituted cycloalkyl, substituted or unsubstituted heterocyclyl, substituted or unsubstituted alkylaryl, substituted or unsubstituted arylalkyl, substituted or unsubstituted aryl, or substituted or unsubstituted heteroaryl, —(CH₂)ₘ—R″′, R″′ represents a hydroxy group, substituted or unsubstituted carbonyl group, an aryl, a cycloalkyl ring, a cycloalkenyl ring, a heterocycle, or a polycycle; and m is zero or an integer ranging from 1 to 8. When E is oxygen, sulfonate cannot be attached to another chemical species, such as to form an oxygen-oxygen bond, or other unstable bonds, as understood by one of ordinary skill in the art.

The term “sulfamoyl” refers to a sulfonamide or sulfonamide represented by the formula

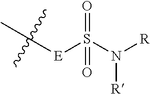

wherein E is absent, or E is substituted or unsubstituted alkyl, substituted or unsubstituted alkenyl, substituted or unsubstituted alkynyl, substituted or unsubstituted aralkyl, substituted or unsubstituted alkylaryl, substituted or unsubstituted cycloalkyl, substituted or unsubstituted aryl, substituted or unsubstituted heteroaryl, substituted or unsubstituted heterocyclyl, wherein independently of E, R and R′ each independently represent a hydrogen, substituted or unsubstituted alkyl, substituted or unsubstituted alkenyl, substituted or unsubstituted alkynyl, substituted or unsubstituted carbonyl, substituted or unsubstituted cycloalkyl, substituted or unsubstituted heterocyclyl, substituted or unsubstituted alkylaryl, substituted or unsubstituted arylalkyl, substituted or unsubstituted aryl, or substituted or unsubstituted heteroaryl, —(CH2)ₘ—R″′, or R and R′ taken together with the N atom to which they are attached complete a heterocycle having from 3 to 14 atoms in the ring structure; R″′ represents a hydroxy group, substituted or unsubstituted carbonyl group, an aryl, a cycloalkyl ring, a cycloalkenyl ring, a heterocycle, or a polycycle; and m is zero or an integer ranging from 1 to 8. In preferred embodiments, only one of R and R′ can be a carbonyl, e.g., R and R′ together with the nitrogen do not form an imide.

The term “sulfoxide” is represented by the formula

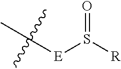

wherein E is absent, or E is alkyl, alkenyl, alkynyl, aralkyl, alkylaryl, cycloalkyl, aryl, heteroaryl, heterocyclyl, wherein independently of E, R represents a hydrogen, substituted or unsubstituted alkyl, substituted or unsubstituted alkenyl, substituted or unsubstituted alkynyl, substituted or unsubstituted amine, substituted or unsubstituted cycloalkyl, substituted or unsubstituted heterocyclyl, substituted or unsubstituted alkylaryl, substituted or unsubstituted arylalkyl, substituted or unsubstituted aryl, or substituted or unsubstituted heteroaryl, —(CH₂)ₘ—R″′, or E and R taken together with the S atom to which they are attached complete a heterocycle having from 3 to 14 atoms in the ring structure; R″′ represents a hydroxy group, substituted or unsubstituted carbonyl group, an aryl, a cycloalkyl ring, a cycloalkenyl ring, a heterocycle, or a polycycle; and m is zero or an integer ranging from 1 to 8.

The term “phosphonyl” is represented by the formula

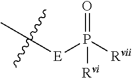

wherein E is absent, or E is substituted or unsubstituted alkyl, substituted or unsubstituted alkenyl, substituted or unsubstituted alkynyl, substituted or unsubstituted aralkyl, substituted or unsubstituted alkylaryl, substituted or unsubstituted cycloalkyl, substituted or unsubstituted aryl, substituted or unsubstituted heteroaryl, substituted or unsubstituted heterocyclyl, wherein, independently of E, R^{vi} and R^{vii} are independently hydrogen, substituted or unsubstituted alkyl, substituted or unsubstituted alkenyl, substituted or unsubstituted alkynyl, substituted or unsubstituted carbonyl, substituted or unsubstituted cycloalkyl, substituted or unsubstituted heterocyclyl, substituted or unsubstituted alkylaryl, substituted or unsubstituted arylalkyl, substituted or unsubstituted aryl, or substituted or unsubstituted heteroaryl, —(CH₂)ₘ—R″′, or R and R′ taken together with the P atom to which they are attached complete a heterocycle having from 3 to 14 atoms in the ring structure; R″′ represents a hydroxy group, substituted or unsubstituted carbonyl group, an aryl, a cycloalkyl ring, a cycloalkenyl ring, a heterocycle, or a polycycle; and m is zero or an integer ranging from 1 to 8.

The term “substituted phosphonyl” represents a phosphonyl in which E, R^{vi} and R^{vii} are independently substituted. Such substituents include, but are not limited to, halogen, azide, alkyl, aralkyl, alkenyl, alkynyl, cycloalkyl, hydroxyl, carbonyl (such as a carboxyl, alkoxycarbonyl, formyl, or an acyl), silyl (such as trimethylsilyl), ether, ester, thiocarbonyl (such as a thioester, a thioacetate, or a thioformate), alkoxyl, phosphoryl, phosphate, phosphonate, phosphinate, amino (or quarternized amino), amido, amidine, imine, cyano, nitro, azido, sulfhydryl, alkylthio, sulfate, sulfonate, sulfamoyl, sulfoxide, sulfonamido, sulfonyl, heterocyclyl, alkylaryl, haloalkyl, —CN, aryl, heteroaryl, and combinations thereof.

The term “phosphoryl” defines a phoshonyl in which E is absent, oxygen, alkoxy, aroxy, substituted alkoxy or substituted aroxy, as defined above, and independently of E, R^{vi} and R^{vii} are independently hydroxyl, alkoxy, aroxy, substituted alkoxy or substituted aroxy, as defined above.

When E is oxygen, the phosphoryl cannot be attached to another chemical species, such as to form an oxygen-oxygen bond, or other unstable bonds, as understood by one of ordinary skill in the art. When E, R^{vi} and R^{vii} are substituted, the substituents include, but are not limited to, halogen, azide, alkyl, aralkyl, alkenyl, alkynyl, cycloalkyl, hydroxyl, carbonyl (such as a carboxyl, alkoxycarbonyl, formyl, or an acyl), silyl (such as trimethylsilyl), ether, ester, thiocarbonyl (such as a thioester, a thioacetate, or a thioformate), alkoxyl, phosphoryl, phosphate, phosphonate, phosphinate, amino (or quarternized amino), amnido, amidine, imine, cyano, nitro, azido, sulfhydryl, alkylthio, sulfate, sulfonate, sulfamoyl, sulfoxide, sulfonamido, sulfonyl, heterocyclyl, alkylaryl, haloalkyl, —CN, aryl, heteroaryl, and combinations thereof.

The term “polyaryl” refers to a chemical moiety that includes two or more fused aryl groups. When two or more fused heteroaryl groups are involved, the chemical moiety can be referred to as a “polyheteroaryl.”

The term “substituted polyaryl” refers to a polyaryl in which one or more of the aryls are substituted, with one or more substituents including, but not limited to, halogen, azide, alkyl, aralkyl, alkenyl, alkynyl, cycloalkyl, hydroxyl, carbonyl (such as a carboxyl, alkoxycarbonyl, formyl, or an acyl), silyl (such as trimethylsilyl), ether, ester, thiocarbonyl (such as a thioester, a thioacetate, or a thioformate), alkoxyl, phosphoryl, phosphate, phosphonate, phosphinate, amino (or quarternized amino), amido, amidine, imine, cyano, nitro, azido, sulfhydryl, alkylthio, sulfate, sulfonate, sulfamoyl, sulfoxide, sulfonamido, sulfonyl, heterocyclyl, alkylaryl, haloalkyl, —CN, aryl, heteroaryl, and combinations thereof. When a polyheteroaryl is involved, the chemical moiety can be referred to as a “substituted polyheteroaryl.”

The term “C₃-C₂₀ cyclic” refers to a substituted or unsubstituted cycloalkyl, substituted or unsubstituted cycloalkenyl, substituted or unsubstituted cycloalkynyl that have from three to 20 carbon atoms, as geometric constraints permit. The cyclic structures are formed from single or fused ring systems. The substituted cycloalkyls, cycloalkenyls, and cycloalkynyls are substituted as defined above for the alkyls, alkenyls, and alkynyls, respectively.

The terms “hydroxyl” and “hydroxy” are used interchangeably and are represented by —OH.

The terms “thiol” and “sulfhydryl” are used interchangeably and are represented by —SH.

The term “oxo” refers to ═O.

The terms “cyano” and “nitrile” are used interchangeably to refer to —CN.

The term “nitro” refers to —NO₂.

The term “phosphate” refers to —O—PG₃.

The term “azide” or “azido” are used interchangeably to refer to —N₃.

### II. Compositions

An injectable formulation for treating cancers shows improved safety and efficacy of delivery of exatecan or its pharmaceutically acceptable salt when administered to tumors such as cutaneous cancerous tumors and tumors in other sites. In the preferred embodiment, the exatecan is dissolved in distilled water at a pH in which the exatecan, preferably exatecan mesylate, is soluble, so that when administered to a tumor, the pH of the tumor causes the exatecan to precipitate within the tumor, thereby causing higher concentrations of the exatecan as well as longer retention within the tumor. The exatecan can be administered intratumorally and/or peritumorally. Co-injection of immunostimulatory agents with exatecan mesylate enhances anti-tumor activity. In another preferred embodiment, the exatecan encapsulated in particles for sustained release, local delivery of the exatecan. The formulation can be used to treat peritoneal carcinomatosis that develops from metastasis of gastrointestinal and/or gynecological cancers including, but not limited to, appendiceal cancer, gastric cancer, ovarian cancer, pancreatic cancer, cervical cancer, uterine cancer, etc.

### A. Exatecans

The exatecan has a structure:

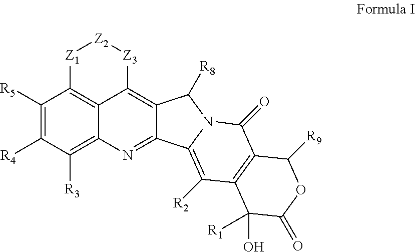

wherein:
R₁ is unsubstituted C₁-C₆ alkyl or substituted C₁-C₆ alkyl (such as substituted with one or more groups selected from halogen, hydroxyl, thiol, nitro, cyano, amino (—NH₂), C₁-C₃ alkyl, C₁-C₃ alkylamino, C₁-C₃ dialkylamino, C₁-C₃ alkoxy, and combinations thereof), R₂, R₃, R₄, R₅, R₈, and R₉ are independently hydrogen, halogen, hydroxyl, thiol, nitro, cyano, amino (—NH₂), unsubstituted C₁-C₆ alkyl, substituted C₁-C₆ alkyl, unsubstituted C₁-C₆ alkenyl, substituted C₁-C₆ alkenyl, unsubstituted C₁-C₆ alkynyl, substituted C₁-C₆ alkynyl, unsubstituted C₁-C₆ alkylamino, substituted C₁-C₆ alkylamino, unsubstituted C₁-C₆ dialkylamino, substituted C₁-C₆ dialkylamino, unsubstituted C₁-C₆ alkoxy, substituted C₁-C₆ alkoxy, unsubstituted C₁-C₆ alkylthio, substituted C₁-C₆ alkylthio, unsubstituted carbonyl, substituted carbonyl, unsubstituted amide, substituted amide, unsubstituted aryl, substituted aryl, unsubstituted polyaryl, substituted polyaryl, unsubstituted heteroaryl, substituted heteroaryl, unsubstituted polyheteroaryl, substituted polyheteroaryl, unsubstituted C₃-C₁₀ cycloalkyl, substituted C₃-C₁₀ cycloalkyl, unsubstituted cyclohexyl, or substituted cyclohexyl,
Z₁, Z₂, and Z₃ are independently O, S, or —CR₆R₇, wherein R₆ and R₇ are independently hydrogen, halogen, hydroxyl, thiol, nitro, cyano, amino (—NH₂), unsubstituted C₁-C₆ alkyl, substituted C₁-C₆ alkyl, unsubstituted C₁-C₆ alkenyl, substituted C₁-C₆ alkenyl, unsubstituted C₁-C₆ alkynyl, substituted C₁-C₆ alkynyl, unsubstituted C₁-C₆ alkylamino, substituted C₁-C₆ alkylamino, unsubstituted C₁-C₆ dialkylamino, substituted C₁-C₆ dialkylamino, unsubstituted C₁-C₆ alkoxy, substituted C₁-C₆ alkoxy, unsubstituted C₁-C₆ alkylthio, substituted C₁-C₆ alkylthio, unsubstituted carbonyl, substituted carbonyl, unsubstituted amide, substituted amide, unsubstituted aryl, substituted aryl, unsubstituted polyaryl, substituted polyaryl, unsubstituted heteroaryl, substituted heteroaryl, unsubstituted polyheteroaryl, substituted polyheteroaryl, unsubstituted C₃-C₁₀ cycloalkyl, substituted C₃-C₁₀ cycloalkyl, unsubstituted cyclohexyl, or substituted cyclohexyl, and
wherein at least one of Z₁, Z₂, and Z₃ is —C(R₆)(NR₁₀R₁₁), wherein R₁₀ and R₁₁ are independently hydrogen, unsubstituted C₁-C₆ alkyl, substituted C₁-C₆ alkyl, unsubstituted C₁-C₆ alkenyl, substituted C₁-C₆ alkenyl, unsubstituted C₁-C₆ alkynyl, substituted C₁-C₆ alkynyl, unsubstituted carbonyl, substituted carbonyl, unsubstituted aryl, substituted aryl, unsubstituted polyaryl, substituted polyaryl, unsubstituted heteroaryl, substituted heteroaryl, unsubstituted polyheteroaryl, substituted polyheteroaryl, unsubstituted C₃-C₁₀ cycloalkyl, substituted C₃-C₁₀ cycloalkyl, unsubstituted cyclohexyl, or substituted cyclohexyl, preferably wherein R₁₀ and R₁₁ are hydrogen.

In some forms, the exatecan has a structure as shown in Formula I above, except that R₁ is unsubstituted C₁-C₆ alkyl or substituted C₁-C₆ alkyl (such as substituted with a halogen).

In some forms, the exatecan has a structure as shown in Formula I above, except that R₁ is unsubstituted C₁-C₆ alkyl (such as ethyl).

In some forms, the exatecan has a structure as shown in Formula I above, except that R₂, R₈, and R₉ are hydrogen.

In some forms, the exatecan has a structure as shown in Formula I above, except that R₃, R₄, and R₅ are independently hydrogen, halogen, hydroxyl, thiol, nitro, cyano, amino (—NH₂), unsubstituted C₁-C₆ alkyl, substituted C₁-C₆ alkyl, unsubstituted C₁-C₆ alkenyl, substituted C₁-C₆ alkenyl, unsubstituted C₁-C₆ alkylamino, substituted C₁-C₆ alkylamino, unsubstituted C₁-C₆ dialkylamino, substituted C₁-C₆ dialkylamino, unsubstituted C₁-C₆ alkoxy, substituted C₁-C₆ alkoxy, unsubstituted carbonyl, substituted carbonyl, unsubstituted amide, substituted amide, unsubstituted aryl, substituted aryl, unsubstituted polyaryl, substituted polyaryl, unsubstituted heteroaryl, substituted heteroaryl, unsubstituted polyheteroaryl, substituted polyheteroaryl, unsubstituted C₃-C₁₀ cycloalkyl, substituted C₃-C₁₀ cycloalkyl, unsubstituted cyclohexyl, or substituted cyclohexyl.

In some forms, the exatecan has a structure as shown in Formula I above, except that R₃, R₄, and R₅ are independently hydrogen, halogen, hydroxyl, nitro, cyano, amino (—NH₂), unsubstituted C₁-C₆ alkyl, substituted C₁-C₆ alkyl, unsubstituted C₁-C₆ alkenyl, substituted C₁-C₆ alkenyl, unsubstituted C₁-C₆ alkylanino, substituted C₁-C₆ alkylamino, unsubstituted C₁-C₆ dialkylamino, substituted C₁-C₆ dialkylamino, unsubstituted C₁-C₆ alkoxy, substituted C₁-C₆ alkoxy, unsubstituted carbonyl, substituted carbonyl, unsubstituted amide, substituted amide, unsubstituted aryl, substituted aryl, unsubstituted polyaryl, or unsubstituted polyaryl.

In some forms, the exatecan has a structure as shown in Formula I above, except that R₃, is hydrogen.

In some forms, the exatecan has a structure as shown in Formula I above, except that R₄, and R₅ are independently halogen, unsubstituted C₁-C₆ alkyl, or substituted C₁-C₆ alkyl.

In some forms, the exatecan has a structure as shown in Formula I above, except that the exatecan has the structure:

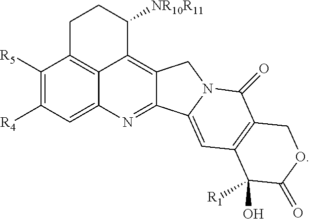

wherein:
R₁₀ and R₁ are independently hydrogen, unsubstituted C₁-C₆ alkyl, substituted C₁-C₆ alkyl, unsubstituted C₃-C₁₀ cycloalkyl, substituted C₃-C₁₀ cycloalkyl, unsubstituted cyclohexyl, or substituted cyclohexyl, preferably wherein R₁₀ and Ru are hydrogen.

In some forms, the exatecan has a structure as shown in Formula I above, except that the exatecan has the structure:

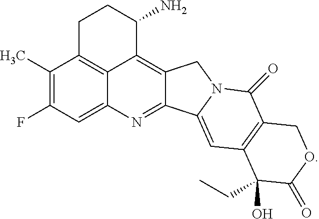 [SMILES: CC[C@@]1(O)C(=O)OCc2c1cc1n(c2=O)Cc2c-1nc1cc(F)c(C)c3c1c2[C@@H](N)CC3]

In some forms, pharmaceutically acceptable salt is formed from organic or inorganic acids selected from the group consisting of sulfonic acids (such as methanesulfonic acid), hydrochloric acid, hydrobromic acid, sulfuric acid, acetic acid, phosphoric acid, citric acid, anhydrous citric acid, maleic acid, mandelic acid, succinic acid, and combinations thereof, i.e., an exatecan salt formed by mixing an exatecan with one or more of these acids. Preferably, the pharmaceutically acceptable salt is an exatecan sulfonate, such as exatecan mesylate.

### B. Carriers

The exatecan or a pharmaceutically acceptable salt thereof is preferably formulated in water at a pH and ionic strength at which the exatecan is soluble. Preferably, the exatecan or the pharmaceutically acceptable salt thereof is dissolved in distilled water. Generally, the pH of the formulation is between about 6.5 and about 7.1, between about 6.5 and 6.9, or between about 6.5 and about 6.84.

C. Immunostimulatory agents

In some forms, the formulations contain or are co-administered with immunostimulatory agents. The immunostimulatory agents can be immunostimulatory oligonucleotides, bacterial lipopolysaccharides (e.g., monophosphoryl lipid A (MPL, SmithKline Beecham)), saponins including QS21 (SmithKline Beecham), interleukins, interferon, CD40 agonists, and cytokines. Examples of immunostimulatory oligonucleotides include those nucleotide sequences containing a CpG motif (a sequence containing an unmethylated cytosine followed by guanosine and linked by a phosphate bond). Bacterial double stranded RNA or oligonucleotides containing palindromic or poly(dG) sequences have also been shown to be innunostimulatory.

The CpG's can include nucleotide modifications/analogs such as phosphorothioate modifications and can be double-stranded or single-stranded. Optionally, the guanosine can be replaced with an analog such as 2′-deoxy-7-deazaguanosine. See Kandimalla, et al., “Nucleic Acids Research 31: 2393-2400, 2003; Recent Pat Inflamm Allerger Drug Discov. 5(1):87-93 (2011); WO02/26757; and WO99/62923 for examples of analog substitutions. The adjuvant effect of CpG oligonucleotides is further discussed in Krieg, Nature Medicine (2003) 9(7): 831-835; McCluskie, et al., FEMS Immunology and Medical Microbiology (2002) 32:179-185; WO98/40100; U.S. Pat. Nos. 6,207,646; 6,239,116 and 6,429,199. The CpG sequence can be directed to Toll-like receptor (TLR9), such as the motif GTCGTT or TTCGTT. See Kandimalla, et al., “Toll-like receptor 9: modulation of recognition and cytokine induction by novel synthetic CpG DNAs”, Biochemical Society Transactions (2003) 31 (part 3): 654-658. The CpG sequence can be specific for inducing a Th1 immune response, such as a CpG-A ODN, or it can be more specific for inducing a B cell response, such a CpG-B ODN. CpG-A and CpG-B ODNs are discussed in Blackwell, et al., J. Immunol. 170: 4061-4068, 2003; Krieg, TRENDS in Immunology 23: 64-65, 2002, and WO01/95935. In some forms, the CpG oligonucleotide can be constructed so that the 5′ end is accessible for receptor recognition. Optionally, two CpG oligonucleotide sequences can be attached at their 3′ ends to form “immunomers”. See, for example, Kandimalla, et al., BBRC 306: 948-95, 2003; Kandimalla, et al., Biochemical Society Transactions 31: 664-658, 2003; Bhagat, et al., BBRC 300: 853-861, 2003, and WO03/035836.

### D. Particles

Also described are particles, preferably core-shell particles, containing an exatecan as described above for Formula I, a hydrophobic core, and a shell, coating, or corona containing hyperbranched polyglycerol. A shell, coating, or corona, as used herein, refers to a distinct outer layer from the core of the particles. The shell, coating, or corona contains hyperbranched polyglycerol and partially or completely surrounds the core. Shell and coating are used interchangeably. The hyperbranched polyglycerol of the shell or coating is covalently bonded to a polymer in the core of the particles; the hyperbranched polyglycerol of the corona is not covalently bonded to a polymer in the core of the particles. In some forms, the exatecan is dispersed within the core of the particles. Preferably, some or all of the hydroxyl groups of the hyperbranched polyglycerol are converted to reactive functional groups to adhere the particles to tissue, and the hyperbranched polyglycerol is covalently bound to the hydrophobic core or polymers forming the core. The reactive functional groups are selected from aldehydes, amines, oximes, and O-substituted oximes. In some forms, some or all of the reactive functional groups are reacted with a poly(ethylene glycol). The data in the examples demonstrate that the particles are effective in treating proliferative disorders, such as cancers, and demonstrate enhanced retention at the site of delivery compared to exatecan administered in its unencapsulated form.

i. Core

The core of the particles is formed of or contains one or more hydrophobic or more hydrophobic materials, such as one or more polymeric materials (e.g., homopolymer, copolymer, terpolymer, etc.). The material may be biodegradable or non-biodegradable. In some forms, the one or more materials are one or more biodegradable polymers. Preferably, a hydrophobic material, such as a hydrophobic polymer, is distributed throughout the core.

In general, synthetic polymers are preferred, although natural polymers may be used and have equivalent or even better properties, especially some of the natural biopolymers which degrade by hydrolysis, such as some of the polyhydroxyalkanoates. Examples of classes of suitable hydrophobic polymers include polyesters (such as polyhydroxyacids), polyanhydrides, poly(ortho)esters, poly(p-dioxanone), poly(polyurethane), polycarbonate, polyphosphate, polyphosphonate, and a combination thereof. Preferably, the hydrophobic polymers include polyesters, preferably linear aliphatic polyesters. Specific examples of suitable hydrophobic polymers include, but are not limited to, poly(lactic acid), poly(glycolic acid), poly(lactic acid-co-glycolic acid), poly(caprolactone), poly(pentadecalactone), poly(hydroxybutyrate-co-hydroxyvalerate), poly(hydroxybutyrate), polybutylene succinate, and a combination thereof. Preferably, the polymeric particles contain poly(lactic acid).

Other materials may also be incorporated including lipids, fatty acids, and phospholipids. These may be dispersed in or on the particles, or interspersed with the polyglycerol coatings discussed below.

ii. Shell

The particles described herein contain a shell, coating, or corona containing hyperbranched polyglycerol (HPG). Hyperbranched polyglycerol is a highly branched polyol containing a polyether scaffold. Hyperbranched polyglycerol can be prepared using techniques known in the art. It can be formed from controlled etherification of glycerol via cationic or anionic ring opening multi-branching polymerization of glycidol. For example, an initiator having multiple reactive sites is reacted with glycidol in the presence of a base to form hyperbranched polyglycerol (HPG). Suitable initiators include, but are not limited to, polyols, e.g., triols, tetraols, pentaols, or greater and polyamines, e.g., triamines, tetraamines, pentaamines, etc. In some forms the initiator is 1,1,1-trihydroxymethyl propane (THP).

Preferably, the surface properties of the HPG are tuned based on the chemistry of hydroxyl groups (such as vicinal diols). For example, the surface properties can be tuned to provide adhesive (sticky) particles, i.e., particles that adhere to the surface of tissues, for example, due to the presence of one or more reactive functional groups, such as aldehydes, amines, oximes, or O-substituted oximes that can be prepared from the vicinal hydroxyl moieties. In these forms, some or all of the hydroxyl groups of the hyperbranched polyglycerol are converted to reactive functional groups to adhere the particles to tissue. Preferred reactive functional groups are aldehydes. In some forms, the surface properties can be turned to provide targeting (e.g., non-covalent targeting) by the introduction of one or more targeting moieties which can be conjugated directly or indirectly to the vicinal hydroxyl moieties. Indirectly refers to transformation of the hydroxy groups to reactive functional groups that can react with functional groups on molecules to be attached to the surface, such as active agents and/or targeting moieties, etc. As discussed above, In some forms, chemical moieties within or between HPG molecules are not crosslinked with themselves, i.e., the HPG is non-crosslinked.

The hyperbranched nature of the polyglycerol allows for a much higher density of hydroxyl groups, reactive functional groups, and/or targeting moieties than polyethylene glycol. For example, the particles described herein can have a density of surface functionality (e.g., hydroxyl groups, reactive functional groups, and/or targeting moieties) of at least about 1, 2, 3, 4, 5, 6, 7, or 8 groups/nm².

The molecular weight of the HPG can vary. For example, in those embodiments wherein the HPG is covalently attached to the materials or polymers that form the core, the molecular weight can vary depending on the molecular weight and/or hydrophobicity of the core materials. The molecular weight of the HPG is generally from about 1,000 to about 1,000,000 Daltons, from about 1,000 to about 500,000 Daltons, from about 1,000 to about 250,000 Daltons, or from about 1,000 to about 100,000 Daltons. In those embodiments wherein the HPG is covalently bound to the core materials, the weight percent of HPG of the copolymer is from about 1% to about 50%, such as about 1, 2, 3, 4, 5, 10, 15, 20, 25, 30, 35, 40, 45 or 50%.

iii. Sheddable polyethylene glycol (PEG) coatings

The HPG-coated particles can be modified by covalently attaching PEG to the surface. This can be achieved by converting some or all of the HPG hydroxyl groups (e.g., vicinyl diol groups) to reactive functional groups, such as (of aldehydes, amines, oximes, and O-substituted oximes, preferably aldehydes) and then reacting the reactive functional groups with functional groups on PEG, such as aliphatic amines, aromatic amines, hydrazines and thiols. The linker has end groups such as aliphatic amines, aromatic amines, hydrazines, thiols and O-substituted oxyamines. The bond inserted in the linker can be disulfide, orthoester and peptides sensitive to proteases.

PEG with a functional group or a linker can form a bond with a reactive functional group on HPG (e.g., aldehyde on PLA-HPGALD) and reverse the bioadhesive (sticky) state of the particles (e.g., PLA-HPGALD) to stealth state. This bond or the linker is labile to pH change or high concentration of peptides, proteins and other biomolecules. “ALD” denotes an aldehyde. After administration systematically or locally, the bond attaching the PEG to the particle can be reversed or cleaved to release the PEG in response to environment and expose the bioadhesive particles (e.g., PLA-HPGALD) to the environment. Subsequently, the particles will interact with the tissue and attach the particles to the tissues or extracellular materials such as proteins. The environment can be acidic environment in tumors, reducing environment in tumors, protein rich environment in tissues.

iv. Drug Loading and Particle Size

Preferably, the exatecan is dispersed within the core of the particles, and is in its free base form. To achieve encapsulation of exatecan in its free base form, a solution containing exatecan (such as exatecan in its salt form) can be contacted with a base, such as an organic base. Without wishing to be bound by theory, it is believed that the base interacts with a counter ion, facilitating formation of exatecan in its free base form. Preferred organic bases are those that contain a nitrogen atom. In some forms, the particles have a higher proportion of the exatecan encapsulated within the particles than on the surface of the particles. In other forms, the exatecan is encapsulated within the particles and is not on the surface of the particles. The exatecan can have a loading between 1% wt/wt and 25% wt/wt, between 1% wt/wt and 20% wt/wt, between 1% wt/wt and 15% wt/wt, or between 5% wt/wt and 15% wt/wt, such as about 10% wt/wt as measured by high-performance liquid chromatography. Preferably, the basis for the weight measurements is the weight of the particles.

The particles can have an average diameter between 1 nm and 1 mm, between 10 nm and 500 nm, between 50 nm and 250 nm, between 50 nm and 200 nm, or between 50 nm and 150 nm, as measured by dynamic light scattering. Preferred diameters are those between 50 nm and 250 nm.

### III. Methods of Making and Reagents Therefore

### A. Exatecan

Methods of making the formulations generally involve dissolving (i) an exatecan or a pharmaceutically acceptable salt thereof, optionally including an imnunostimulatory agent, in water at a pH and concentration at which the exatecan is soluble, most preferably distilled water. In some forms, the solution may be heated to about 40° C. to enhance dissolution of the exatecan or a pharmaceutically acceptable salt thereof. Additional details are provided in the following non-limiting examples.

### B. Particles Containing Exatecan

Particles containing an exatecan, as described above, can be manufactured via a single emulsion-solvent evaporation method. Making the particles containing an exatecan typically involves contacting a solution containing exatecan (such as exatecan in its salt form) with a base. This contacting can be performed by adding a base to a solution containing exatecan. The base can be an organic base. Without wishing to be bound by theory, it is believed that the base interacts with a counter ion, facilitating formation of exatecan in its free base form. Preferred organic bases are those that contain a nitrogen atom. Examples of organic bases include alkylamines (such as triethylamine, diethylamine, methylamine, di-n-butylamine), pyridine, imidazole, benzimidazole, guanidine, anistidines, benzylaniline, methylurea, phenylurea, piperidine, etc.

Preferably, the solution contains a copolymer containing hyperbranched polyglycerol covalently bound to a hydrophobic polymer. Suitable hydrophobic polymers include polyesters (such as polyhydroxyacids), polyanhydrides, poly(ortho)esters, poly(p-dioxanone), poly(polyurethane), polycarbonate, polyphosphate, polyphosphonate, and a combination thereof. Preferred hydrophobic polymers include polyesters, such as linear aliphatic polyesters. Specific examples of suitable hydrophobic polymers include, but are not limited to, poly(lactic acid), poly(glycolic acid), poly(lactic acid-co-glycolic acid), poly(caprolactone), poly(pentadecalactone), poly(hydroxybutyrate-co-hydroxyvalerate), poly(hydroxybutyrate), polybutylene succinate, and a combination thereof. Preferably, the copolymer is a copolymer of poly(lactic acid)-hyperbranched polyglycerol. Preferably, after adding the base, an emulsion is formed from the solution, and volatile organic components are removed (e.g., evaporated) from the emulsion.

For example, the copolymer is dissolved in a suitable organic solvent, such as methylene chloride or dichloromethane (DCM). Exatecan is dissolved in a suitable organic solvent, such as dimethyl formamide (DMF), and an organic base, such as an alkylamine is added to the DMF solution. The polymer and drug solutions are combined and sonicated. An aqueous solvent (e.g., distilled water) is added to a separate container, and then the organic phase (containing the copolymer and exatecan) are added dropwise with vortexing. The glass tube can be cooled on an ice bath, and the mixture sonicated to form an emulsion. The resulting emulsion can be further diluted with an aqueous solvent (e.g., distilled water). Volatile organic components can be removed (e.g., evaporated), and the suspension centrifuged to separate particles.

The surface of the particles can be modified by resuspending the particles and converting some or all of the hydroxyl groups (e.g., vicinal hydroxyl groups) of the hyperbranched polyglycerol to reactive groups such as aldehydes, amines, oximes, and O-substituted oximes. Some or all of the reactive functional groups can be reacted with a poly(ethylene glycol) to introduce sheddable poly(ethylene glycol) on the surface of the particles.

A non-limiting example of how to make particles containing an exatecan is described in Example 7 below.

### IV. Methods of Using

The exatecan, a pharmaceutically acceptable salt thereof, or particles containing an exatecan (preferably core-shell particles described above) can be administered alone, concurrently, or in combination with an immunostimulatory agent. The exatecan, a pharmaceutically acceptable salt thereof, or particles containing an exatecan can be administered concurrently or successively with an immunostimulatory agent in separate formulations.

The exatecan or a pharmaceutically acceptable salt thereof is formulated in water so it stays in solution in the acidic microenvironment of tumor tissues, but precipitates as it moves into normal tissue, thereby increasing the concentration of exatecan in the tumor and decreasing toxicity in adjacent normal tissue. The precipitated exatecan or a pharmaceutically acceptable salt thereof can further serve as a depot, for longer term release into tumor tissues. The exatecan or a pharmaceutically acceptable salt thereof is administered at doses effective to inhibit growth of and/or kill tumor cells, but sufficiently low to avoid or minimize the toxicity of exatecan to healthy tissue.

The particles containing an exatecan can be similarly administered at doses effective to treat a proliferative disorder or disease.

### A. Disorders to be Treated

Exatecan, a pharmaceutically acceptable salt thereof, or particles containing an exatecan can be used to treat a proliferative disorder or disease. In some forms, the proliferative disorder or disease involves cancer cells, stromal cells (e.g., fibroblasts), epithelial cells, endothelial cells (e.g., blood vessel cells), adipose cells, endometrial cells, endocrine cells, blood cells, bone cells, bone marrow cells, muscle cells, brain cells, etc. Non-transformed over proliferative disorders to be treated include endometriosis, fibroids and scarring. Even though endometriosis is not cancer treatment is difficult and the disorder is debilitating and painful.

In particular, the formulations are designed to kill cancer cells and/or inhibit cancer cell growth/proliferation and/or metastasis. Some cancers include skin cancer, esophageal cancer, bladder cancer, ovarian cancer. The formulations are also designed to treat peritoneal carcinomatosis that develops from metastasis of gastrointestinal and/or gynecological cancers. Non-limiting examples of cancers that can give rise to peritoneal carcinomatosis include, but are not limited to, appendiceal cancer, gastric cancer, ovarian cancer, pancreatic cancer, cervical cancer, uterine cancer, etc.

The methods involving exatecan or pharmaceutically acceptable salt thereof typically include administering to a subject in need thereof a therapeutically effective amount of the injectable formulation to kill tumor cells or limit proliferation or metastasis thereof. In the preferred embodiment, the formulation is injected using needles, although it may be possible to administer using a microneedle patch. The precise dosage will vary according to a variety of factors such as subject-dependent variables (e.g., age, immune system health, etc.), the stage of the cutaneous cancer, the volume of the tumor, and/or the treatment being performed. These factors can also determine the volume of formulation to be injected. The formulation containing exatecan or pharmaceutically acceptable salt thereof can be injected into a tumor or tissue adjacent to or abutting the tumor.

The particles containing exatecan can be formulated for administration through one or more routes selected from intraperitoneal, intratumoral, intravenous, intradermal, intraarterial, intralesional, intracranial, intrathecal, intraarticularly, intraprostatic, intraovarian, intrapleural, intratracheal, intravitreal, intramuscular, subcutaneous, subconjunctival, intravesicular, intrapericardial, intraumbilical, by injection, and by infusion.

In a preferred embodiment, the exatecan or particles containing an exatecan are used to treat cutaneous tumors. Cutaneous cancers include basal cell carcinoma, melanoma, squamous cell carcinoma, cutaneous T-cell lymphoma, cutaneous B-cell lymphoma, Merkel cell carcinoma, sebaceous carcinoma, Kaposi sarcoma, and dermatofibrosarcoma protuberans.

The most common cutaneous malignancies are basal cell carcinoma, squamous cell carcinoma, and melanoma. Other cancers include primary cutaneous lymphoma, Merkel cell carcinoma, Kaposi sarcoma, metastatic cancers to the skin. Cutaneous malignancies vary widely in their aggressiveness, morbidity, and mortality. Basal cell and squamous cell carcinomas are the most common cutaneous malignancies. Malignant melanoma is less common and is much more aggressive.

Primary cutaneous lymphomas are a heterogeneous group of non-Hodgkin lymphomas that affect the skin and may progress to systemic disease. These have replaced Hodgkin disease as the most common adult lymphomas, and they are more common among the black population than the white population and are more common in men than in women.

The majority of cases are cutaneous T-cell lymphomas, comprising 65% to 92% of cutaneous lymphomas, of which mycosis fungoides and Sézary syndrome are most common. Mycosis fungoides has more of an indolent behavior, and Sézary syndrome frequently is more aggressive. The tumors of mycosis fungoides can become very large and may be mushroom-shaped, thus the termfungoides. A smaller subset of cutaneous lymphomas includes primary cutaneous CD4+small/medium pleomorphic lymphoma, cutaneous γδ T-cell lymphoma, primary cutaneous anaplastic large cell lymphoma, and cutaneous B-cell lymphoma. The T cells affected are those responsible for skin homing.

The cutaneous lymphomas present a diagnostic challenge, because they can mimic several other dermatologic diseases, including psoriasis, contact dermatitis, nummular eczema, atopic dermatitis, lichen simplex chronicus, lymphoid contact dermatitis, and tinea corporis. Differentiation of cutaneous lymphomas and other conditions is done through historical information, clinical presentation, and histopathologic analysis.

Cutaneous malignancies vary widely in their aggressiveness, morbidity, and mortality. Basal cell and squamous cell carcinomas are by far the most common cutaneous malignancies. Malignant melanoma is less common and is much more aggressive. Although they are uncommon, it is important to have the ability to recognize these pathologies. The following malignancies are included: primary cutaneous lymphomas, Merkel cell carcinoma, Kaposi sarcoma, adnexal tumors, and metastatic cancers to the skin.

Cutaneous malignant neoplasms refer to the broader group of skin growths that are cancerous.

Merkel cell carcinoma (MCC) is a rare and highly aggressive cutaneous neuroendocrine small-cell malignancy. It is highly metastatic to the regional lymphatic basin, as well as nodal and hematogenous spread, and it is often fatal with a 33% mortality rate. MCC also is known as apudoma, primary neuroendocrine carcinoma of the skin, primary small cell carcinoma of the skin, and trabecular carcinoma of the skin. The etiology includes UV solar radiation exposure, immunosuppression, advanced age, and Merkel cell polyomavirus (MCPyV). Kaposi sarcoma (KS) is a rare disease and is believed to be a virally induced angioproliferative disorder associated with human herpesvirus 8 (HHV-8). HHV-8 is required for the development of KS; however, cofactors such as an immunocompromised state also are necessary for KS development. The four subtypes of KS are classic, AIDS-associated, endemic, and immunosuppression-associated. Apocrine and adnexal carcinomas are endocrine mucin-producing tumors that are rare and very commonly are misdiagnosed. This is due to the general infrequency of each of these conditions, as well as similar features to more common skin conditions. Cutaneous metastasis of cancers originating in other organs can occur by hematologic or lymphatic embolization, or direct implantation during surgical procedures.

Many primary tumors have the potential to metastasize to the skin and subcutaneous tissues. These include breast cancer most commonly, but it also can occur with esophageal, gastric, or colon cancers, nasopharyngeal cancer, lymphomas, pancreatic cancer, renal cell carcinoma, lung cancer, and ovarian cancer.

### B. Dosages

The formulation containing exatecan, pharmaceutically acceptable salt thereof, or particles containing an exatecan is administered to deliver an effective amount of an exatecan or a pharmaceutically acceptable salt thereof, to (i) kill cancer cells and/or inhibit cancer cell growth at the site of injection, (ii) inhibit a topoisomerase, such as topoisomerase I, (iii) reduce activation of a topoisomerase (e.g., topoisomerase I) pathway, (iv) inhibit DNA replication in a cancer cell, or a combination thereof.

The effective amount of the exatecan or a pharmaceutically acceptable salt thereof, can be ascertained from assays investigating the (i) killing cancer cells and/or inhibition cancer cell growth, (ii) inhibition of a topoisomerase, such as topoisomerase I, (iii) reduction in activation of a topoisomerase (e.g., topoisomerase I) pathway, (iv) inhibition of DNA replication in a cancer cell, or a combination thereof, compared to a control that does not contain the exatecan or a pharmaceutically acceptable salt thereof. In some forms, the exatecan or a pharmaceutically acceptable salt thereof, has a half-maximal inhibitory concentration (ICso) of inhibiting a topoisomerase (e.g. DNA topoisomerase 1) of less than 1,000 μM, less than 100 μM, less than 10 μM, less than 1 μM, less than 0.1 μM, less than 0.01 μM, or less than 0.001 μM; for example, 0.001 μM-1,000 μM, 0.001 μM-100 μM, 0.001 μM-10 μM, 0.01 μM-1,000 μM, 0.01 μM-100 μM, 0.0 μM-10 μM, 0.1 μM-1,00 μM, 0.1 μM-100 μM, 0.1 μM-10 μM, 1 μM 1,000 μM, 1 μM-100 μM, 1 μM-10 μM, or any subrange or specific number therebetween. In some forms, the concentration of the exatecan or a pharmaceutically acceptable salt thereof in the formulation is less than 30 mM, less than 25 mM, less than 20 mM, less than 15 mM, less than 10 mM, less than 5 mM, or less than 1 mM; for example, 0.001 mM-30 mM, 0.01 mM-30 mM, 0.1 mM-25 mM, 0.1 mM-30 mM, 1.0 mM-20 mM, 5 mM 20 mM, 10 mM-20 mM, or any subrange or specific number therebetween. In some forms, the dose of the exatecan or a pharmaceutically acceptable salt thereof in the formulation can be expressed in terms of mass of drug/mass of a subject in need thereof. Suitable doses include less than 55 mg/kg; less than 45 mg/kg; less than 40 mg/kg; less than 35 mg/kg; less than 30 mg/kg; less than 25 mg/kg; less than 20 mg/kg; less than 15 mg/kg; less than 10 mg/kg; or less than 5 mg/kg, such as between 0.5 mg/kg and 50 mg/kg; between 1.25 mg/kg and 45 mg/kg; between 1.25 mg/kg and 40 mg/kg; between 1.25 mg/kg and 35 mg/kg; between 1.25 mg/kg and 30 mg/kg; between 1.25 mg/kg and 25 mg/kg; between 1.25 mg/kg and 20 mg/kg; between 1.25 mg/kg and 15 mg/kg; between 1.25 mg/kg and 10 mg/kg; between 1.25 mg/kg and 5 mg/kg; about 10 mg/kg, about 5 mg/kg, about 2.5 mg/kg or about 1.25 mg/kg.

In some forms, the dose of the immunostimulatory agent in the formulation can be expressed in terms of mass of immunostimulatory agent/mass of a subject in need thereof. Suitable doses include than less than 15 mg/kg; than less than 10 mg/kg; less than 5 mg/kg; less than 2.5 mg/kg; less than 2 mg/kg; less than 1.5 mg/kg; or less than 1 mg/kg; such as between 0.1 mg/kg and 10 mg/kg; between 0.1 mg/kg and 5 mg/kg; between 0.1 mg/kg and 2.5 mg/kg; between 0.1 mg/kg and 2 mg/kg; between 0.1 mg/kg and 1.5 mg/kg; between 0.1 mg/kg and 1 mg/kg; about 5 mg/kg, about 2.5 mg/kg or about 0.5 mg/kg.

The formulation may be administered as a single dose or in multiple doses at appropriate intervals throughout the course of treatment. Administering multiple doses can involve injecting a first dose and waiting for a suitable time to elapse before injecting a second dose. For multiple doses, subsequent doses can be the same dose level as a prior dose, or can be modified depending on the tumor response or any associated toxicity. Suitable times can be one day, two days, three days, four days, five days, one week, two weeks, three weeks, one month, etc. Determining effective doses and appropriate intervals can be ascertained by those of skill in the art with nothing more than routine experimentation.

The present invention will be further understood by reference to the following non-limiting examples.

### EXAMPLES

### Example 1: Intratumoral Injection of Exatecan

### Materials and Methods

### (i) Exatecan Preparation

Exatecan mesylate (“EXA”) (DX895If; MedChemExpress #HY-13631A) was dissolved in sterile dH₂0 (Invitrogen 10977-023) to prepare a 5 mg/mL or a 10 mg/mL stock solution. At 5 mg/mL, the exatecan mesylate dissolves immediately. To prepare the 10 mg/mL stock, warming to 60° C. plus sonication and vortexing were needed. The concentration was confirmed by HPLC and further dilutions prepared as needed in sterile dH2O.

### (ii) Experimental Design

SCC tumors were established by subcutaneous injection of 5 million PDVC57 cells into the right flank of syngeneic C57BL/6J mice. Seven days later, when tumors ranged from 4.5-5.5 mm in diameter, mice were evenly distributed among the treatment groups (N=5-10 mice/group, see below) and received intratumoral injections of vehicle (dH₂O) or exatecan.

(iii) Intratumoral Delivery

A 0.5-mL 31 G syringe (BD Insulin Syringe #328468) was used to deliver 0.1 mg or 0.2 mg exatecan in a 20-microliter volume, or 0.4 mg in a 40-microliter volume (because of solubility limitations). Control groups received an equal volume of sterile dH₂O. The needle was placed into the tumor and the appropriate volume was slowly injected (approximately over 5 sec). The needle was held in place for 10 seconds after the injection was complete, then slowly removed while maintaining finger pressure on the injection site.

The mice received a single injection of 0.2 mg or 0.4 mg exatecan. Alternatively, mice received 0.1 mg on day 0 and another 0.1 mg on day 3.

### Results

TABLE 1
Results of Experiment 1

| | | | Mice | |
|---|---|---|---|---|
| | | | remaining | |
| | | Histology | (days post- | Tumor |
| Group (N) | Toxicity | (N) | treatment) | incidence |
| Vehicle: | None | Tumor in | 0 | 100% |
| dH₂O (10) | observed | 10/10 | | |
| EXA: 0.2 mg | None | Tumor in 2 | 4 (day 53) - | 20% |
| (10) | observed | of 6 mice | no palpable | |
| | | harvested (4 | tumor | |
| | | mice were | | |
| | | not | | |
| | | harvested) | | |

16 days post-treatment, when the first vehicle control tumors reached 1 cm in one dimension, half (N=5) of each group was harvested for histology: tumors were measured, ranked by volume (V=(½)(W²×L)), and every-other one harvested (#1, 3, 5, 7, and 9 were harvested and #2, 4, 6, 8, and 10 were not).

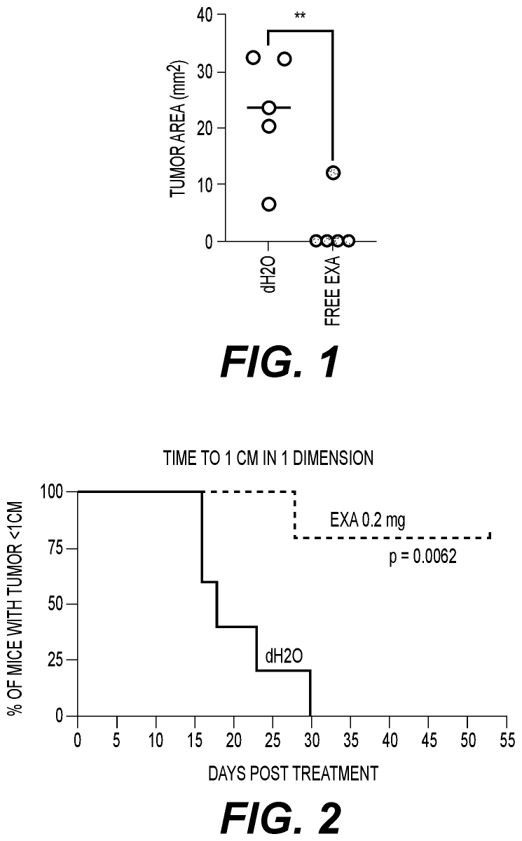

FIG. 1 shows histologic tumor area for mice harvested on day 16.

The remaining Experiment 1 mice (N=5/group) continued to be monitored for tumor growth and were harvested if their tumor reached 1 cm in one dimension. All five vehicle-treated mice had tumors that progressed to 1 cm. Only one of the remaining five exatecan-treated mice had a tumor that progressed to 1 cm. The other four exatecan-treated mice had no palpable tumor 53 days post-treatment. The results are shown in FIG. 2.

TABLE 2
Experiment 2

| | | | Mice | |
|---|---|---|---|---|
| | | | remaining | |
| | | Histology | (days post- | Tumor |
| Group (N) | Toxicity | (N) | treatment) | incidence |
| Vehicle: | None | Tumor in 5/5 | 0 | 100% |
| dH₂O (5) | observed | | | |
| EXA: 0.1 mg | Lethal in 2 of | Tumor in 4 | 2 (day 36) - | 4 of 7 |
| ×2 (9) | 9 mice | of 5 mice | no palpable | 57% |
| | | harvested | tumor | |
| | | (2 mice were | | |
| | | not | | |
| | | harvested) | | |
| EXA: 0.4 mg | Lethal in 7 of | N/A | 1 (day 36) - | |
| (8) | 8 mice | | no palpable | |
| | | | tumor | |

In summary, exatecan was very effective treatment at a 0.2-mg dose per animal and was well tolerated. The exatecan showed lethality at 0.4 mg per animal. Two doses of 0.1 mg per animal separated by 3 days was less well tolerated and less effective than the single 0.2 mg dose.

Based on these observations, it is possible that anti-tumor efficacy is driven by local Cₘₐₓ concentrations of drug and that broader systemic toxicity is driven by exposure over time, that can be determined by an area-under-curve analysis. These findings demonstrate strategies for dosing regimens that allow for the effective local use of exatecan via intratumoral/peritumoral injection.

### Example 2: pH-Dependent Exatecan Mesylate Solubility

Materials and methods A water-based formulation was made containing exatecan mesylate at a concentration of 10 mg/mL exatecan mesylate in water (Invitrogen UltraPure Distilled Water #10977-015). Phosphate buffers A and B were made by dissolving either 1M potassium phosphate monobasic (KH₂PO₄) for buffer A or 1M potassium phosphate dibasic (KH₂PO₄) for buffer B in distilled water. Solutions with pH values ranging between 4.3 and 9.6 were prepared by mixing buffer A (pH 4.3) with buffer B (pH 9.6) at pre-determined percentages. A pH meter was used to confirm the pH of the resulting phosphate buffer solutions by dipping the electrode into the solution.

To determine the pH solubility of the water-based formulation, 5 μL of the 10 mg/mL EXA in DI water was added to 100 μL 1M phosphate buffer at varying pH levels in a 96-well plate to produce a final concentration of −0.5 mg EXA per milliliter. The solubility tests were conducted at pH 4.30 (0% buffer B), pH 5.6 (10% buffer B), pH 6.1 (20% buffer B), pH 6.4 (30% buffer B), pH 6.7 (40% buffer B), pH 7.0 (50% buffer B), pH 7.2 (60% buffer B), pH 7.5 (70% buffer B), pH 7.8 (80% buffer B), pH 8.2 (90% buffer B) and pH 9.6 (100% buffer B). Precipitation was assessed and images of the precipitate at the bottom of the 96-well plate were taken after 1 hour.

### Results

Precipitation following addition of the EXA/DI water mixture to the phosphate buffer was generally immediate. Table 3 shows the results from qualitative assessment of the precipitate formed following addition of EXA/DI water to phosphate buffer solutions of varying pH levels. Generally, little to no differences were observed in exatecan mesylate solubility in DI water alone compared to various acidic phosphate buffer solutions as shown in Table 3. As shown in Table 3, at a pH of 7.12 and higher, exatecan mesylate shows enhanced precipitation and a corresponding decrease in solubility. In addition, the size of the resulting precipitate is correlated with the pH level; higher pH caused precipitation to be aggressive, causing fine precipitate to form, whereas lower pH resulted in slower precipitation resulting in coarse solids. These results demonstrate the effect of pH on the solubility of exatecan mesylate.

TABLE 3
pH-dependent solubility of exatecan mesylate.

| | Buffer | Buffer | | Precipitate rating^{a} |
|---|---|---|---|---|
| Sample # | A (%) | B (%) | pH | (Yes/No) |
| Control | 0 | 0 | 7 | No |
| (distilled water) | | | | |
| 1 | 100 | 0 | 4.3 | No |
| 2 | 90 | 10 | 5.6 | No |
| 3 | 80 | 20 | 6.1 | No |
| 4 | 70 | 30 | 6.4 | No |
| 5 | 60 | 40 | 6.7 | Yes |
| 6 | 50 | 50 | 7.0 | Yes |
| 7 | 40 | 60 | 7.2 | Yes |
| 8 | 30 | 70 | 7.5 | Yes |
| 9 | 20 | 80 | 7.8 | Yes |
| 10 | 10 | 90 | 8.2 | Yes |
| 11 | 0 | 100 | 9.6 | Yes |

^{a}Precipitate rating “Yes” means visible precipitate observed at 10× magnification, and “No” means no visible precipitate observed at 10× magnification.

### Example 3: In Vivo Testing of a Water-Based Formulation Containing Exatecan Mesylate in a Mouse Model of SCC

### Materials and Methods

### (i) Animals

Six-week old female C57BL/6J mice were used in this study.

### (ii) PDVC57 Injection

PDVC57 cells were developed in, and obtained from, the laboratory of Dr. Allan Balmain (Quintanilla, et al., Carcinogenesis 1991, 12 (10), 1875-1881). Mice were anesthetized with avertin and their flanks shaved. The mice were then given an intradermal injection with of 5 million PDVC57 cells in suspension in 100 μL PBS. A 1 cc syringe fitted with a 27 g needle was used for the intradermal injection.

(iii) Drug Treatment

Seven days following tumor cell inoculation, the mice were given either a single intratumoral injection of 10 mg/mL exatecan mesylate in ultrapure distilled water (Invitrogen, Catalog #10977-015) or a vehicle injection using a BD insulin syringe (30-gauge×8 mm needle; #328468). The vehicle was invitrogen UltraPure Distilled Water #10977-015. The treatment volume was 0.02 mL.

### (iv) Tumor Measurements

Mice were sedated. Calipers were used to measure tumor diameter. Tumor volume was calculated from micrometer measurements using the following formula:

$$Volume , V = ( \frac{1}{2} ) ( {W}^{2} \times L ) )$$

### (v) Tumor Processing and Histology

When tumors reached the endpoint, i.e., tumor volume of 1 cm³, mice were euthanized by isoflurane inhalation and tumors dissected away from subcutaneous tissue. Isolated tumors were bisected along the longest axis, fixed in 10% neutral buffered fonnalin and submitted to Yale Research Pathology for embedding, sectioning, and H&E staining. Resulting slides were scanned using a Zeiss AxioObserver Z₁ with TissueGnostics TissueFAXS software. Images were analyzed using Fiji software.

### Results

### (i) Effect of a Single Intratumoral Injection on Tumor Growth

In a first experiment, 20 mice with tumors ranging in size from 45-85 mm³ were examined. The group size was 10. Ten of the mice were given a single injection of a water-based formulation containing exatecan mesylate (10 mg/mL), while the other 10 mice received a vehicle injection of ultrapure DI water and served as the control group. For each group (experimental group or control), half of the mice were harvested 16 days post-treatment for histological assessment, the other half were harvested 32 days post-treatment if they had discernible tumors or monitored until day 66 if no palpable tumors were observed.

Table 4 shows the number of mice that have tumors following intratumoral injection of either Exatecan mesylate in DI water or vehicle (DI water alone). All of the vehicle-treated mice had discernible tumors that persisted 32 days post-treatment (Table 4). Comparably, 80% (8 out of 10) of the Exatecan-treated mice were tumor free when assessed at day 16 and day 66 post-treatment (Table 4). These results indicate that the water-based formulation containing Exatecan mesylate is effective as an injectable treatment for skin-cancer based tumors.

TABLE 4
Effects of a single injection of a water-based formulation
containing exatecan mesylate on the presence of tumors.

| | | | Mice | | |
|---|---|---|---|---|---|
| | | | remaining | | % of mice |
| | Day 16 | Day 32 | (day 66 | Tumor | that |
| | harvest for | harvest for | post- | incidence | eliminated |
| Group (N) | histology | histology | treatment | overall | their tumor |
| Vehicle | Tumor in | Tumor in | 0 | 10 of 10 | 0% |
| (10) | 5 of 5 | 5 of 5 | | (100%) | |
| Exatecan | Tumor in | Tumor in | 4 - no | 2 of 10 | 80% |
| (10) | 1 of 5 | 1 of 1. 4 | palpable | (20%) | |
| | | mice were | tumor | | |
| | | not | | | |
| | | harvested) | | | |

To confirm the data in Table 4, histological assessments were done on 5 of the mice in each group. While discernible tumors were observed in all 5 of the vehicle-treated mice, no discernible tumor was observed in 4 out of 5 of the exatecan-treated mice. Mice treated with a single dose of the water-based formulation containing exatecan mesylate exhibited minimal tissue damage as well as decreased inflammation of surrounding tissue compared to vehicle-treated mice.

### Example 4: In Vivo Testing of a Water-Based Formulation Containing Exatecan Mesylate in a Mouse Model of SCC

### Materials and Methods

In a second experiment, 15 mice with tumors ranging in size from 50-100 mm³, calculated as described above, were examined.

PDVC57 cells were injected into 6-week-old female C57BL/6J mice. Treatment was administered by a single intratumoral injection seven days after tumor cell inoculation. Intratumoral injection was performed using BD insulin syringe (30-gauge×8 mm needle; #328468). The vehicle was Invitrogen UltraPure Distilled Water #10977-015.

The experimental group received 10 mg/mL exatecan. The treatment volume was 0.02 mL. Ten of the mice were given a single injection of a water-based formulation containing exatecan mesylate while the other 5 mice received a vehicle injection of ultrapure DI water and served as the control group. These mice were observed until the endpoint at which their tumor volume was 1 cm³. Clinical images taken on day 22 post-treatment showed large tumors in 5 of 5 vehicle control mice, while only 1 of 10 exatecan-treated mice had a large tumor at this time. This experiment has continued through day 77 post-treatment and the results are provided below.

### Results

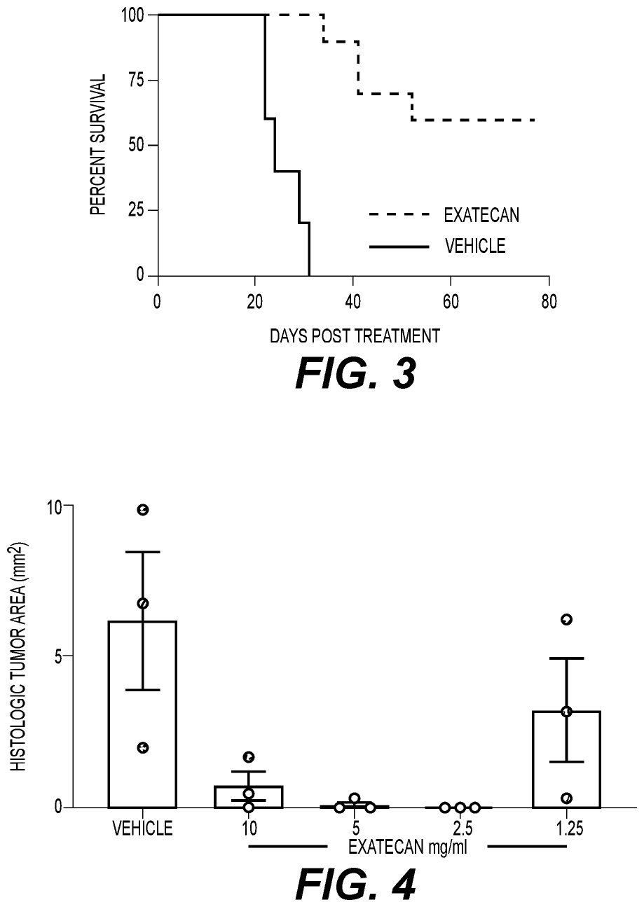

All vehicle control mice (5/5) had tumors that reached endpoint volume (1 cm³) by day 31 post-treatment. Only 4 of 10 exatecan-treated mice had tumors that reached endpoint volume 34-52 days post-treatment. At Day 77 post-treatment, 6 of the 10 exatecan-treated mice remain with no palpable tumor (60% cure). FIG. 3 is a survival curve showing the percent of mice that survived up to 77 days following a single injection of either the vehicle or the water-based formulation containing exatecan mesylate.

### Example 5: Effect of Titrated Doses of a Single Injection of the Water-Based Formulation Containing Exatecan Mesylate on Tumor Size

### Materials and Methods

In a third experiment, 15 mice (i.e., five groups each containing three mice) with tumors ranging in size 59-80 mm³, calculated as described above, were examined. PDVC57 cells were injected into 6-week-old female C57BL/6J mice. Treatment was administered seven days after tumor cell inoculation. Intratumoral injection performed using BD insulin syringe (30-gauge×8 mm needle; #328468). Mice were randomly assigned to one of five groups and were given a single intratumoral injection of either the vehicle (UltraPure Distilled Water, Invitrogen, #10977-015) or one of the following concentrations of the water-based formulation containing exatecan mesylate: 10 mg/mL, 5 mg/mL, 2.5 mg/mL or 1.25 mg/mL for a total treatment volume of 0.02 mL. All the mice were harvested 14 days post treatment for histological assessments.

### Results

FIG. 4 is a bar graph showing the dose dependent effect of exatecan treatment on tumor size in mice. Mice treated with exatecan, showed reduced tumor size for all doses administered (FIG. 4) compared to vehicle. Mice that received 10 mg/mL exatecan had significantly reduced tumors compared to mice that received 1.25 mg/mL and vehicle-treated mice (FIG. 4). Mice that received 2.5 mg/mL and 5 mg/mL had almost complete reduction in tumor size compared to vehicle-treated mice (FIG. 4).

### Example 6: Effect of Combining Exatecan Mesylate with Immunostimulatory Agent

### Materials and Methods

SCC tumors were established by subcutaneous injection of 5 million PDVC57 cells into the right flank of six-week old female C57BL/6J mice. The mice were partitioned into four groups of 10, and treatment was administered by a single intratumoral injection seven days after tumor cell inoculation. The tumor sizes ranged from 46-80 mm³ volume (V=(M)(W²×L))) calculated from micrometer measurements.

Intratumoral injection was performed using a BD insulin syringe (30 gauge×8 mm needle; #328468). The treatments were as follows:
(1) Vehicle: Invitrogen UltraPure Distilled Water #10977-015;
(2) Exatecan mesylate concentration: 5 mg/ml, dose 5 mg/kg (100 μg/20 g mouse);
(3) CpG ODN 1826 (InvivoGen tlrl-1826-1): 0.5 mg/kg CpG (10 μg/20 g mouse); and
(4) Exatecan mesylate concentration: 5 mg/ml, dose 5 mg/kg (100 μg/20 g mouse)+CpG ODN 1826 (InvivoGen tlrl-1826-1): 0.5 mg/kg CpG (10 μg/20 g mouse).

The treatment volume was 0.02 mL.

In this experiment, mice were weighed to assess treatment toxicity and all mice were followed to endpoint. In this experiment the endpoint was defined as a tumor measuring 1 cm in one dimension.

### Results

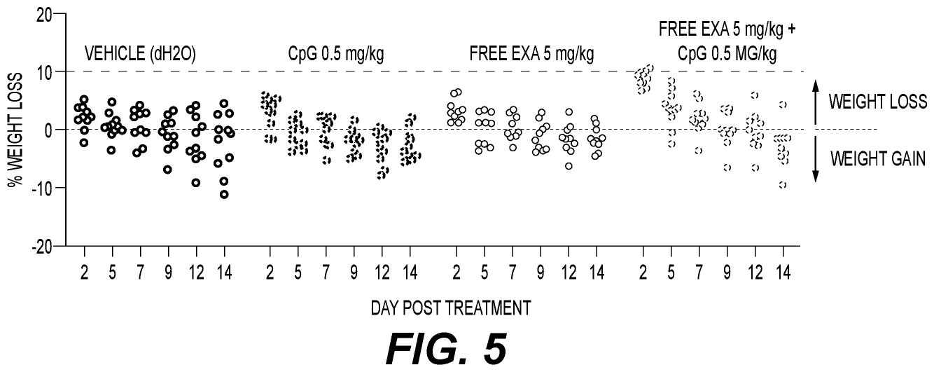

The toxicity results over a 14-day period post-treatment are shown in FIG. 5. Two mice in the Free EXA+CpG group reached 10.0 and 10.5% weight loss, but rapidly recovered. All other mice lost <10% of their bodyweight.

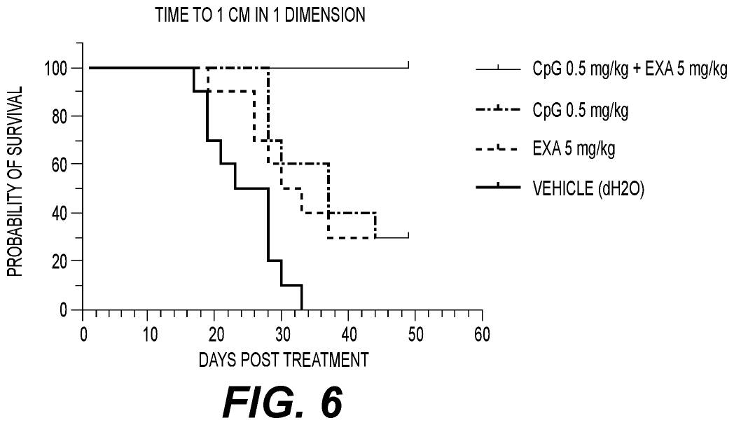

The tumor growth results over a 49-day period post-treatment are shown in FIG. 6. As shown, the combination of free EXA and CpG showed the best probability of survival. The log-rank (Mantel-Cox) test on day 49 post-treatment is shown in Table 5.

TABLE 5
Log-rank (Mantel-Cox) test on day 49 post-treatment

| Tests | p-value |
|---|---|
| Vehicle vs EXA (5 mg/kg) | 0.0217 |
| Vehicle vs CpG (0.5 mg/kg) | 0.0010 |
| Vehicle vs EXA (5 mg/kg) + CpG (0.5 mg/kg) | <0.0001 |
| EXA (5 mg/kg) vs CpG (0.5 mg/kg) | 0.6046 |
| EXA (5 mg/kg) vs EXA (5 mg/kg) + CpG (0.5 mg/kg) | 0.0011 |
| CpG (0.5 mg/kg) vs EXA (5 mg/kg) + CpG (0.5 mg/kg) | 0.0012 |

Based on these results, the water-based (e.g., distilled water) exatecan formulations show major unexpected advantages when delivered intratumorally and/or peritumorally, i.e., injected into and/or around tumors, such as those of the skin, as evidenced by the major efficacy and safety findings in these preclinical models of SCC. Co-injection of immunostimulatory agents the exatecan (e.g., exatecan mesylate) also enhanced anti-tumor activity.

### Example 7: Exatecan Encapsulated in Bioadhesive Nanoparticles

### Materials and Methods

### (i) Preparation of Bioadhesive Nanoparticles (BNPs) Encapsulating Exatecan (BNP-111 Preparation)

45 mg of PLA-HPG polymer was dissolved overnight in 0.7 mL DCM in a glass vial. 10 wt % free base equivalent of exatecan mesylate (SB-111) was added to 0.75 uL DMF in an Eppendorf tube, and 1.2 eq. triethylamine was added, sonicated for 30 s, and let sit for 10 min. Polymer and drug solutions were combined and sonicated for 30 s. In a glass tube, 3 mL of distilled water was added, and organic phase was added dropwise with vortexing. The glass tube was immediately cooled on ice bath, and the mixture was sonicated with a probe sonicator four times for 10 s each with 10 s interval. The resulting emulsion was diluted further in 10 mL distilled water with stirring and transferred to a 100 mL RB flask. Volatiles were removed for 15 min at 75 mbar. Nanoparticles were isolated by centrifugation at 4000 g for 30 min using Amicon ultra-15 centrifugal filter units (100 kDa MWCO), and washed twice with 15 mL distilled water to yield NNP-1 11. Nanoparticles were resuspended at 25 mg nanoparticle per mL concentration for conversion to BNP-111. To the nanoparticle suspension, 1 eq. volume of 10×PBS and 0. M NaIO₄ (aq) was added, and the resulting mixture was incubated on ice for 20 min. The reaction was stopped by adding 1 eq. volume of 0.2 M Na₂SO₃ (aq). Nanoparticles were collected by centrifugation at 4000 g using Amicon ultra-15 centrifugal filter units (100 kDa MWCO), and washed twice with 15 mL distilled water. Resulting BNP-111 was resuspended in DI water and stored at 4° C. until use. Drug concentration was determined by dissolving an aliquot of particles in DMSO and analysis by HPLC.

### (ii) HPLC Protocol

10 uL of nanoparticle suspension was diluted in 90 μL of DMSO and vortexed for 1 min. Further dilution in DMSO was performed to achieve appropriate concentration of approximately 10-200 μg/mL for analysis. HPLC system (Shimadzu) contained a Shimadzu Prominence-I LC-2030C Plus module with LC-2030 UV detector (D2 lamp). A C-18 column (Microsorb-MV 100-5 C18 250×4.6 mm) was used with column temperature at 40° C. with an injection volume of 10 μL and a detection wavelength of 370 nm. The mobile phase was made of an aqueous phase (A) of water with 0.1% (v/v) trifluoroacetic acid (TFA), and an organic phase (B) of acetonitrile with 0.1% (v/v) TFA. Flow was set at 1.0 mL/min with a total run time of 15 min with a gradient of 5% B from 0-2 min, 5-95% B from 2-7 min, 95-100% B from 7-10 min, 100% B from 10-12 min, 100-5% B from 12-12.1 min, and 5% from 12.1-15 min. Drug concentration in BNP-111 was determined relative to a standard curve generated from 10-200 μg/mL SB-111 in DMSO.

(iii) Animal Studies Using BNP-111

### Animals and Housing

C57Bl/6 female (for PDVC57 and ID8) or male (for YUMMER1.7) mice were purchased from The Jackson Laboratory at 6 weeks of age and allowed to acclimate for 5-7 days before use in experiments. Mice were housed under specific pathogen-free conditions in an accredited (Association for Assessment of Laboratory Animal Care) facility with food and water provided ad libitum. All in vivo studies were approved by the Yale Animal Care and Use Committee.

### (A) PDVC57 SCC and YUMMER1.7 Melanoma Models

PDVC57 cells were washed twice in sterile PBS, resuspended in sterile PBS and a 1 ml 27 G syringe used to inject 5 million cells in 100 μl subcutaneously (forming a bleb) into the rear flank of syngeneic female C57Bl/6 mice. Tumor growth was monitored 3×/wk using a calibrated micrometer. Tumor volume was calculated as Volume=(Width²×Length)/2. Seven days after tumor cell injection, when tumor volume ranged from approximately 50-90 mm³, animals were divided into treatment groups with equivalent average tumor volumes.

YUMMER1.7 cells were washed twice in sterile PBS, resuspended in sterile PBS and a lml 27 G syringe used to inject 300,000 cells in 50 μl subcutaneously (forming a bleb) into the rear flank of syngeneic male C57Bl/6 mice. Tumor growth was monitored 3×/wk using a calibrated micrometer. Tumor volume was calculated as Volume=(Width²×Length)/2. Eight days after tumor cell injection, when tumor volume ranged from approximately 50-90 mm³, animals were divided into treatment groups with equivalent average tumor volumes.

### Treatment

Animals received a single intratumoral injection of vehicle (sterile dH₂O, Invitrogen 10977-023), blank BNP or BNP-111 in sterile dH₂O. BNP concentration was adjusted to deliver the desired dose of SB-111 (2.5 mg/kg-15 mg/kg) in a 20 μL volume.

Intratumoral injection: A 0.5 ml 31 G syringe (BD Insulin Syringe #328468) was used to deliver a 20 μL volume. The needle was placed into the tumor and the appropriate volume was slowly injected. The needle was held in place for 10 seconds after the injection, then slowly removed while maintaining finger pressure on the injection site.

### Monitoring

Animals were monitored 3×/wk. Mouse weight was recorded as a measure of systemic toxicity. Tumors were measured 3×/wk using a calibrated micrometer. Tumor volume was calculated as Volume=(Width²×Length)/2.

Endpoints: For histologic studies, animals were euthanized 14 days post-treatment and tumors dissected away from subcutaneous tissue. Tumors were then bisected through the longest plane, fixed in 10% neutral buffered formalin, paraffin embedded, sectioned (6 μm) and stained with hematoxylin and eosin. A Zeiss Axio Observer microscope equipped with TissueGnostics TissueFAXS 7.1 image acquisition software was used to examine tumor cross-sections. Images were analyzed and tumor area quantified using Fiji (NIH) software.

For survival studies, an endpoint of tumor volume=1 cm³ was used.

Statistical analyses were performed with Graphpad Prism 9.0. Student's t-test was used to compare histologic tumor area. Survival curve comparisons used Mantel-Cox test. In all cases significance was established at P<0.05.

### (B) ID8 Ovarian Cancer Model

Luciferase expressing ID8-Luc tumor cells were washed twice in sterile PBS, resuspended in sterile PBS and a 1 ml 27 G syringe used to inject 1 million cells intraperitoneally (i.p.) into syngeneic female C57Bl/6 mice.

### Treatment

Seven days after implantation of ID8-Luc cells, animals received the first of 4 total once-weekly injections of vehicle (sterile dH₂O), BNP-111, or free drug SB-111. BNP-111 concentration was adjusted to deliver the desired dose (1.25-2.5 mg/kg) in 0.5 ml volume.

### Monitoring

Mouse weight was recorded weekly as a measure of systemic toxicity. Tumor growth was monitored once per week by bioluminescent IVIS imaging 10 minutes after 150 mg/kg D-luciferin was administered i.p.

### HPLC Protocol

10 uL of nanoparticle suspension was diluted in 90 μL of DMSO and vortexed for 1 min. Further dilution in DMSO was performed to achieve appropriate concentration of approx. 10-200 μg/mL for analysis. HPLC system (Shimadzu) contained a Shimadzu Prominence-I LC-2030C Plus module with LC-2030 UV detector (D2 lamp). A C-18 column (Microsorb-MV 100-5 C_{18 250×4.6} mm) was used with column temperature at 40° C. with an injection volume of 10 μL and a detection wavelength of 370 nm. The mobile phase was made of an aqueous phase (A) of water with 0.1% (v/v) trifluoroacetic acid (TFA), and an organic phase (B) of acetonitrile with 0.1% (v/v) TFA. Flow was set at 1.0 mL/min with a total run time of 15 min with a gradient of 5% B from 0-2 min, 5-95% B from 2-7 min, 95-100% B from 7-10 min, 100% B from 10-12 min, 100-5% B from 12-12.1 min, and 5% from 12.1-15 min. Drug concentration in BNP-111 was determined relative to a standard curve generated from 10-200 μg/mL SB-111 in DMSO.

### Results

### (i) Particle Size and Drug Loading

The average size of a population of BNP-111 nanoparticles was 132.8±19, with a polydispersity of 0.232±0.034. The loading of the free base exatecan was 10.2±0.75% wt/wt.

### (ii) BNP-111 In Vitro Effect on Tumor Cell Viability

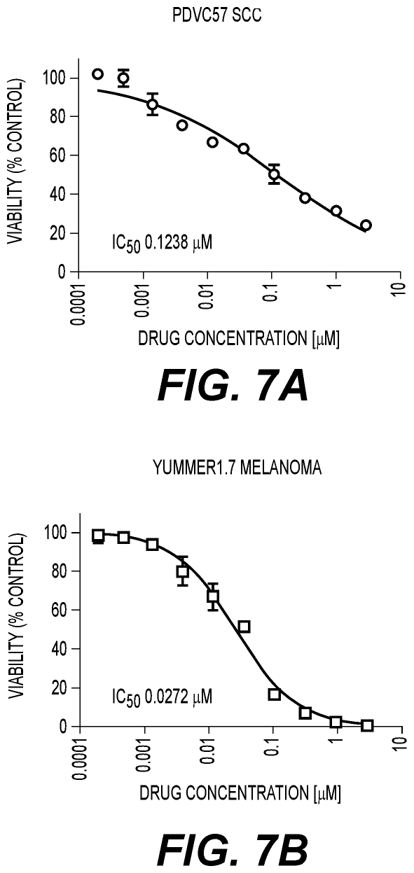

PDVC57 mouse squamous cell carcinoma cells or YUMMER1.7 mouse melanoma cells were plated at 5000 cells/well in triplicate in the presence of titrated amounts of encapsulated drug (BNP-111) and cultured in complete medium for 72 hr, then cell viability was determined using CellTiter-Glo (Promega) and IC₅₀ calculated (Prism). BNP-encapsulated drug efficiently killed both PDVC57 and YUMMER 1.7 tumor cell lines (FIGS. 7A and 7B).

(iii) BNP-111 In Vivo Experiments with PDVC57 SCC Models

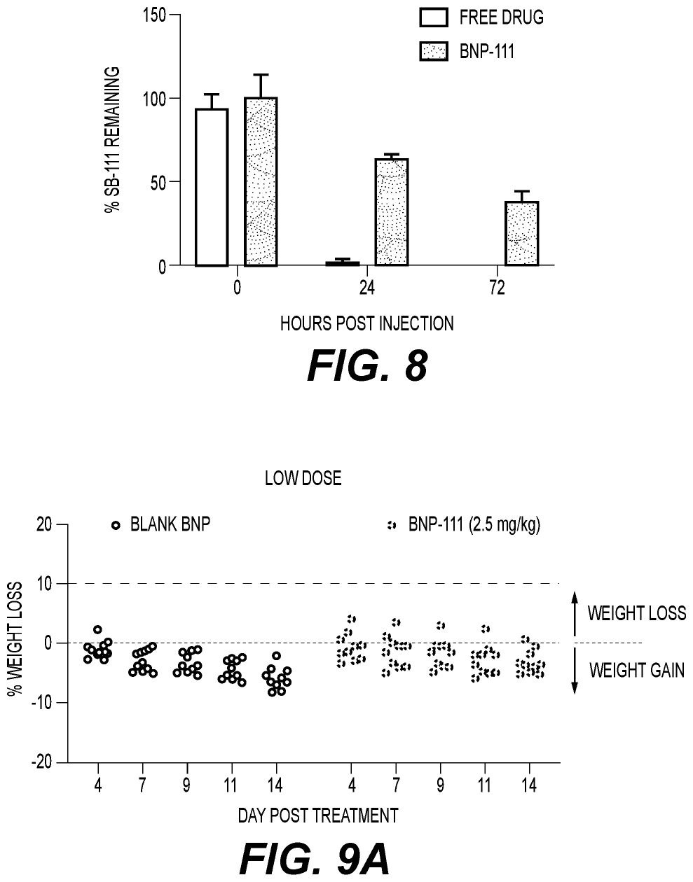

PDVC57 cells (5 million) were subcutaneously implanted into syngeneic female C57Bl/6 mice. Seven days later 0.1 mg free drug (SB-111) or encapsulated drug (BNP-111) was injected into the tumor. Tumors were harvested immediately (0 hr) or 24 hr or 72 hr later and processed to extract the drug, which was quantified by HPLC. BNP encapsulation resulted in prolonged retention of the drug within the tumor, with 37.77% of the injected drug retained in the tumor at 72 hr, whereas only 0.27% free drug was detectable at this time (FIG. 8).

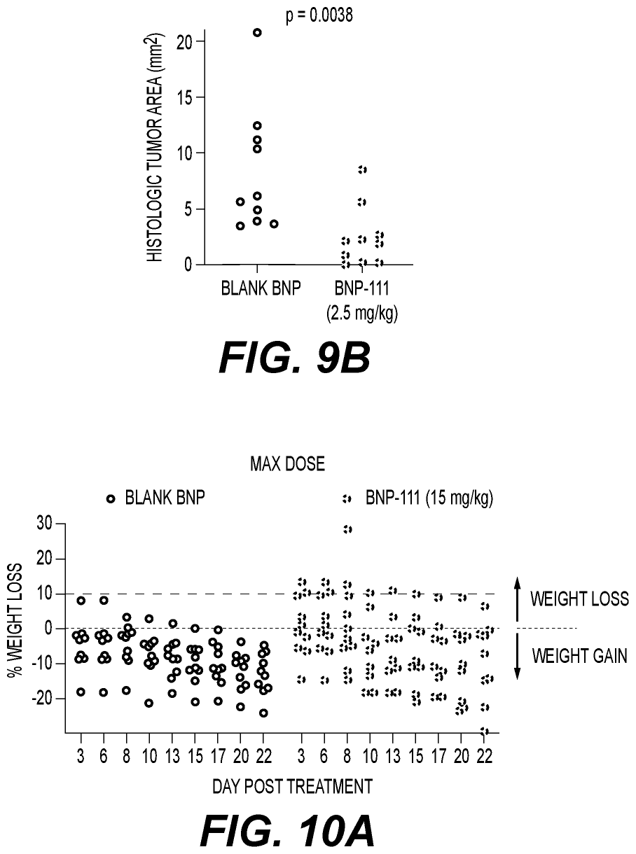

PDVC57 cells (5 million) were subcutaneously implanted into syngeneic female C57Bl/6 mice. Seven days later mice (N=10/group) received a single intra-tumoral injection of low-dose BNP-111 (2.5 mg/kg) or control blank BNP. Mouse weight was recorded throughout the experiment as a measure of systemic toxicity. Tumors were harvested 14 days after BNP-111 injection for histologic tumor area determination (TissueGnostics and ImageJ). BNP-111 (2.5 mg/kg) was well tolerated with no signs of systemic toxicity and resulted in an average 70.39% reduction in histologic tumor area 14 days post-treatment (p=0.0038, Student's t-test) (FIGS. 9A and 9B).

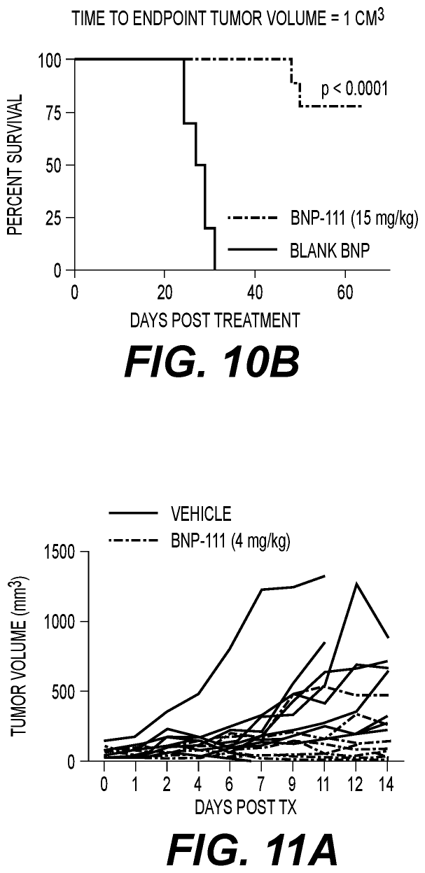

PDVC57 cells (5 million) were subcutaneously implanted into syngeneic female C57Bl/6 mice. Seven days later mice (N=10/group) received a single intra-tumoral injection of maximum dose BNP-111 (15 mg/kg) or control blank BNP. Mouse weight was recorded as a measure of systemic toxicity and tumors were measured 3×/wk with a calibrated micrometer. At max dose 1 of 10 mice receiving BNP-111 lost significant bodyweight and was removed from the study. 100% of control treated mice reached endpoint tumor volume (1 cm³) by 31 days post-treatment, whereas BNP-111 treated mice showed extended survival (p<0.0001), with 77.77% remaining >60 days post-treatment (FIGS. 10A and 10B).

(iv) BNP-111 In Vivo Experiments with YUMMERJ.7 Melanoma Models

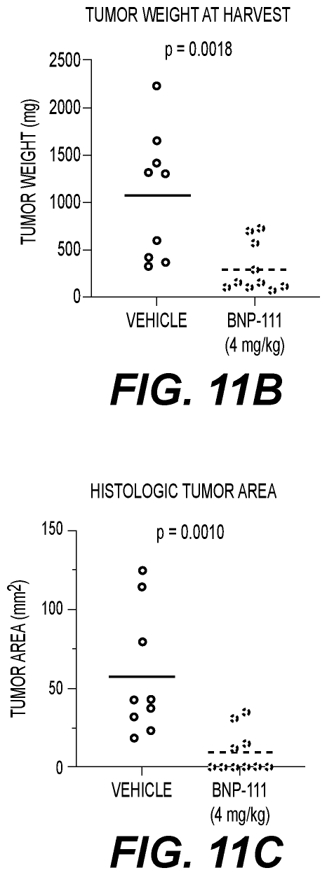

YUMMER1.7 cells (300.000) were subcutaneously implanted into syngeneic male C57Bl/6 mice. Eight days later mice (N=9 or 10/group) received a single intra-tumoral injection of vehicle (dH₂O) or BNP-111 (4 mg/kg). Tumor growth was measured by calibrated micrometer. Tumors were harvested 14 days after treatment, weighed, and formalin fixed for histologic tumor area determination (TissueGnostics and ImageJ). BNP-111 (4 mg/kg) resulted in an average 72.75% reduction in tumor weight (P=0.0018) and 83.88% reduction in histologic tumor area 14 days post-treatment (p=0.0010, Student's t-test) (FIGS. 11A-11C).

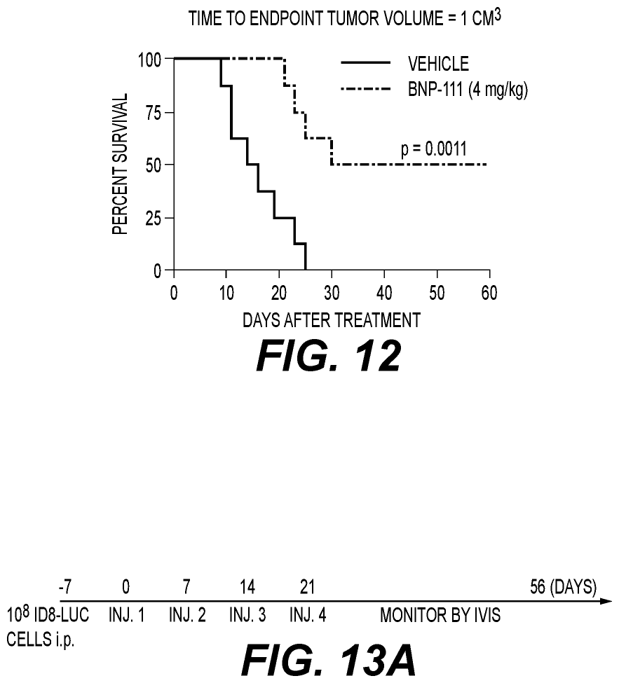

YUMMER1.7 cells (300.000) were subcutaneously implanted into syngeneic male C57Bl/6 mice. Eight days later mice (N=8/group) received a single intra-tumoral injection of vehicle (dH₂O) or BNP-111 (4 mg/kg). Tumors were measured 3×/wk with a calibrated micrometer. 100% of control treated mice reached endpoint tumor volume (1 cm³) by 25 days post-treatment, whereas BNP-111 treated mice showed extended survival (p =0.0011), with 50% remaining >60 days post-treatment (FIG. 12).

(v) BNP-111 In Vivo Experiments with ID8 Ovarian Cancer Models

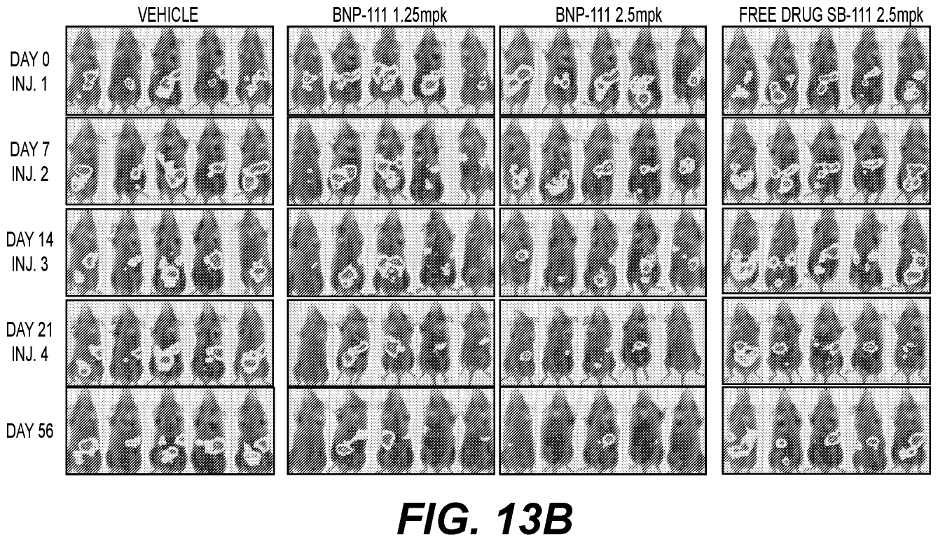

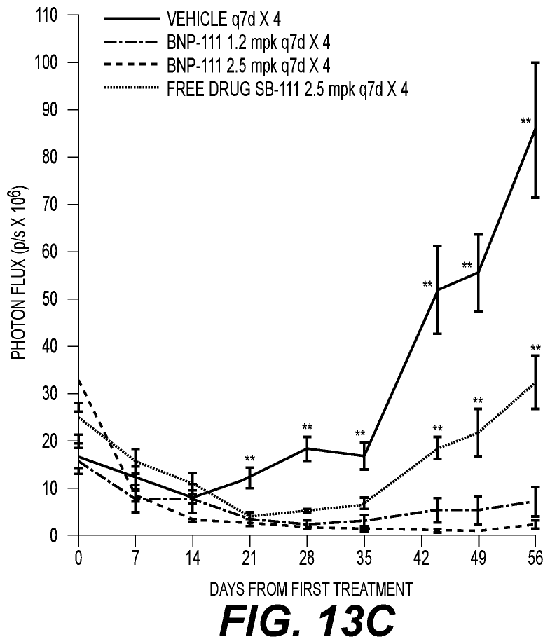

Luciferase expressing ID8-Luc tumor cells (1 million) were implanted i.p. into syngeneic C57Bl/6 mice. Seven days later mice (N=5/group) received the first of 4 once-weekly i.p. injections of vehicle (dH₂O), BNP-111 or free drug (SB-111). Tumor growth was monitored by IVIS and tumor burden (photon flux) graphed. **P<0.01 vs BNP-111. BNP-111 reduced tumor burden at day 56 by 91.60% (1.25 mpk) and 97.43% (2.5 mpk), while free drug SB-111 reduced tumor burden by only 62.16% (2.5 mpk) at this timepoint (FIGS. 13A-13C).

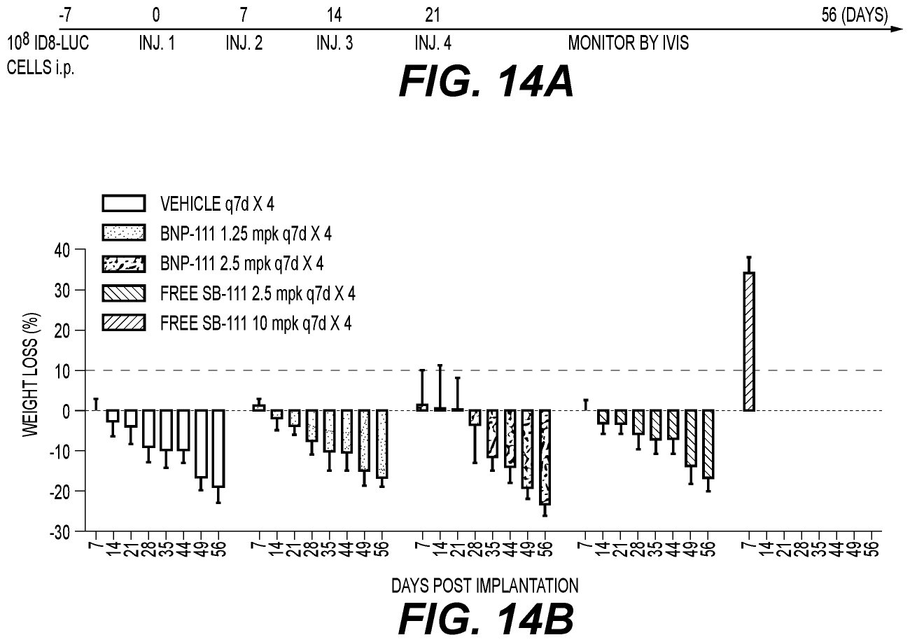

For safety/tolerability studies, luciferase expressing ID8-Luc tumor cells (1 million) were implanted i.p. into syngeneic C57Bl/6 mice. Seven days later mice (N=5 per group) received the first of 4 once-weekly i.p. injections (Tx, treatment) of vehicle (dH₂O), BNP-111 or free drug (SB-111). Mouse weight was recorded throughout the experiment as a measure of systemic toxicity. BNP-111 was well tolerated at 2.5 mg/kg and 1.25 mg/kg q7d x4. Free drug (SB-111) was well tolerated at 2.5 mg/kg q7d x4, but not at a higher dose (FIGS. 14A and 14B).

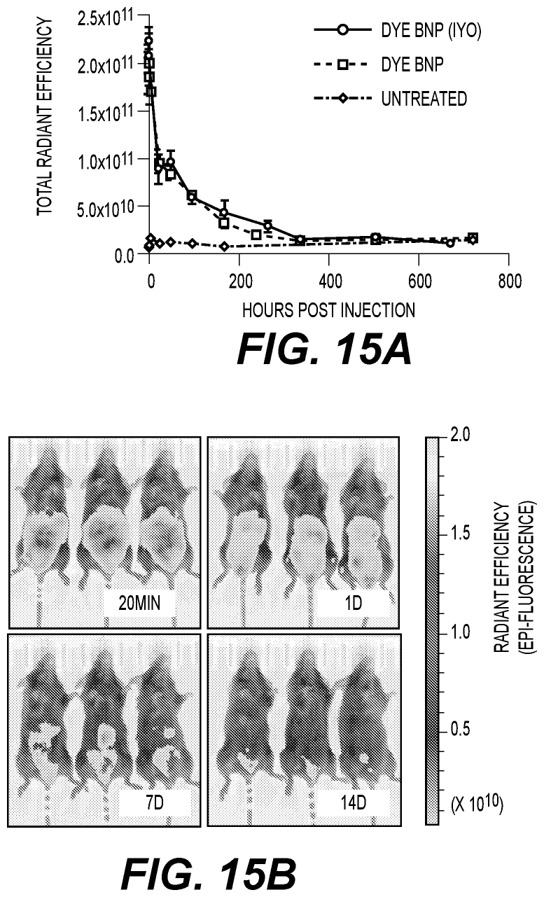

In general, the BNP showed enhanced and persistent adhesion upon IP injection, as shown by the injection of BNPs encapsulating a dye (FIGS. 15A and 15B)

Those skilled in the art will recognize or be able to ascertain using no more than routine experimentation, many equivalents to the specific embodiments of the invention described herein. Such equivalents are intended to be encompassed by the following claims.

1. An injectable formulation for treating or reducing cancers, the formulation comprising an aqueous solution of exatecan or a pharmaceutically acceptable salt thereof.

2. The injectable formulation of claim 1, having a pH at which exatecan is soluble, but which is converted to a pH at which exatecan is not soluble when exposed to the pH of normal tissue.

3. The injectable formulation of claim 1, wherein the exatecan has a structure: 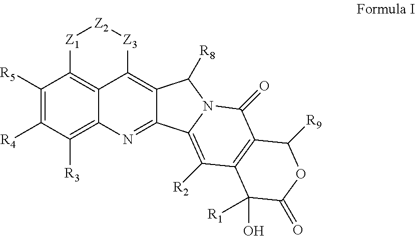
wherein:
R1 is unsubstituted C1-C6 alkyl or substituted C1-C6 alkyl (such as substituted with one or more groups selected from halogen, hydroxyl, thiol, nitro, cyano, amino (—NH2), C1-C3 alkyl, C1-C3 alkylamino, C1-C3 dialkylamino, C1-C3 alkoxy, and combinations thereof), R2, R3, R4, R5, R8, and R9 are independently hydrogen, halogen, hydroxyl, thiol, nitro, cyano, amino (—NH2), unsubstituted C1-C6 alkyl, substituted C1-C6 alkyl, unsubstituted C1-C6 alkenyl, substituted C1-C6 alkenyl, unsubstituted C1-C6 alkynyl, substituted C1-C6 alkynyl, unsubstituted C1-C6 alkylamino, substituted C1-C6 alkylamino, unsubstituted C1-C6 dialkylamino, substituted C1-C6 dialkylamino, unsubstituted C1-C6 alkoxy, substituted C1-C6 alkoxy, unsubstituted C1-C6 alkylthio, substituted C1-C6 alkylthio, unsubstituted carbonyl, substituted carbonyl, unsubstituted amide, substituted amide, unsubstituted aryl, substituted aryl, unsubstituted polyaryl, substituted polyaryl, unsubstituted heteroaryl, substituted heteroaryl, unsubstituted polyheteroaryl, substituted polyheteroaryl, unsubstituted C3-C10 cycloalkyl, substituted C3-C10 cycloalkyl, unsubstituted cyclohexyl, or substituted cyclohexyl,
Z1, Z2, and Z3 are independently O, S, or —CR6R7, wherein R6 and R7 are independently hydrogen, halogen, hydroxyl, thiol, nitro, cyano, amino (—NH2), unsubstituted C1-C6 alkyl, substituted C1-C6 alkyl, unsubstituted C1-C6 alkenyl, substituted C1-C6 alkenyl, unsubstituted C1-C6 alkynyl, substituted C1-C6 alkynyl, unsubstituted C1-C6 alkylamino, substituted C1-C6 alkylamino, unsubstituted C1-C6 dialkylamino, substituted C1-C6 dialkylamino, unsubstituted C1-C6 alkoxy, substituted C1-C6 alkoxy, unsubstituted C1-C6 alkylthio, substituted C1-C6 alkylthio, unsubstituted carbonyl, substituted carbonyl, unsubstituted amide, substituted amide, unsubstituted aryl, substituted aryl, unsubstituted polyaryl, substituted polyaryl, unsubstituted heteroaryl, substituted heteroaryl, unsubstituted polyheteroaryl, substituted polyheteroaryl, unsubstituted C3-C10 cycloalkyl, substituted C3-C10 cycloalkyl, unsubstituted cyclohexyl, or substituted cyclohexyl, and wherein at least one of Z1, Z2, and Z3 is —C(R6)(NR₁₀R11), wherein R10 and R11 are independently hydrogen, unsubstituted C1-C6 alkyl, substituted C1-C6 alkyl, unsubstituted C1-C6 alkenyl, substituted C1-C6 alkenyl, unsubstituted C1-C6 alkynyl, substituted C1-C6 alkynyl, unsubstituted carbonyl, substituted carbonyl, unsubstituted aryl, substituted aryl, unsubstituted polyaryl, substituted polyaryl, unsubstituted heteroaryl, substituted heteroaryl, unsubstituted polyheteroaryl, substituted polyheteroaryl, unsubstituted C3-C10 cycloalkyl, substituted C3-C10 cycloalkyl, unsubstituted cyclohexyl, or substituted cyclohexyl, preferably wherein R10 and R11 are hydrogen.

4. The injectable formulation of claim 3, wherein R1 is unsubstituted C1-C6 alkyl or substituted C1-C6 alkyl (such as substituted with a halogen), optionally wherein R1 is unsubstituted C1-C6 alkyl (such as ethyl).

5. (canceled)

6. The injectable formulation of claim 3, wherein R2, R8, and R9 are hydrogen.

7. The injectable formulation of claim 3, wherein R3, R4, and R5 are independently hydrogen, halogen, hydroxyl, thiol, nitro, cyano, amino (—NH2), unsubstituted C1-C6 alkyl, substituted C1-C6 alkyl, unsubstituted C1-C6 alkenyl, substituted C1-C6 alkenyl, unsubstituted C1-C6 alkylamino, substituted C1-C6 alkylamino, unsubstituted C1-C6 dialkylamino, substituted C1-C6 dialkylamino, unsubstituted C1-C6 alkoxy, substituted C1-C6 alkoxy, unsubstituted carbonyl, substituted carbonyl, unsubstituted amide, substituted amide, unsubstituted aryl, substituted aryl, unsubstituted polyaryl, substituted polyaryl, unsubstituted heteroaryl, substituted heteroaryl, unsubstituted polyheteroaryl, substituted polyheteroaryl, unsubstituted C3-C10 cycloalkyl, substituted C3-C10 cycloalkyl, unsubstituted cyclohexyl, or substituted cyclohexyl.

8. (canceled)

9. The injectable formulation of claim 3, wherein R3 is hydrogen; and/or R4, and R5 are independently halogen, unsubstituted C1-C6 alkyl, or substituted C1-C6 alkyl.

10. (canceled)

11. The injectable formulation of claim 3, wherein the exatecan has a structure: 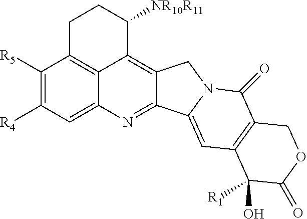
wherein:
R10 and R11 are independently hydrogen, unsubstituted C1-C6 alkyl, substituted C1-C6 alkyl, unsubstituted C3-C10 cycloalkyl, substituted C3-C10 cycloalkyl, unsubstituted cyclohexyl, or substituted cyclohexyl, preferably wherein R10 and R11 are hydrogen.

12. The injectable formulation of claim 3, wherein the exatecan has a structure:

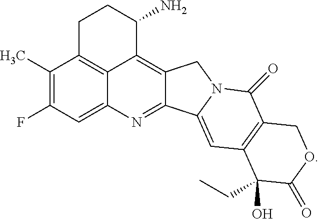 [SMILES: CC[C@@]1(O)C(=O)OCc2c1cc1n(c2=O)Cc2c-1nc1cc(F)c(C)c3c1c2[C@@H](N)CC3]

13. The injectable formulation of claim 1, wherein the pH of the formulation is between about 6.5 and about 7.1, between about 6.5 and 6.9, or between about 6.5 and about 6.8; and/or wherein the injectable formulation comprises between 0.05 mg and 1.5 mg/ml, preferably between 0.5 mg and 1.0 mg/ml, of exatecan or pharmaceutically acceptable salt thereof, in 20 μL of the distilled water.

14. (canceled)

15. (canceled)

16. The injectable formulation of claim 1, further comprising an immunostimulatory agent, optionally wherein the immunostimulatory agents comprise immunostimulatory oligonucleotides, bacterial lipopolysaccharides, saponins, interleukins, interferon, CD40 agonists, cytokines, or a combination thereof.

17. (canceled)

18. (canceled)

19. Particles comprising:
(i) an exatecan,
(i) a hydrophobic core, and
(ii) a shell, coating, or corona comprising hyperbranched polyglycerol,
wherein some or all of the hydroxyl groups of the hyperbranched polyglycerol are converted to reactive functional groups to adhere the particles to tissue,
wherein the hyperbranched polyglycerol is covalently bound to the hydrophobic core or polymers forming the core, and
wherein the reactive functional groups are selected from the group consisting of aldehydes, amines, oximes, and O-substituted oximes, optionally wherein some or all of the reactive functional groups are reacted with a poly(ethylene glycol).

20. The particles of claim 19, wherein the core comprises a hydrophobic polymer.

21. The particles of claim 20, wherein the hydrophobic polymer is selected from the group consisting of poly(lactic acid), poly(glycolic acid), and copolymers thereof.

22. The particles of claim 19, wherein the one or more reactive functional groups are aldehydes.

23. The particles of claim 19, wherein some or all of the reactive functional groups are reacted with a poly(ethylene glycol).

24. The particles of claim 19, wherein the exatecan is dispersed within the core of the particles.

25. The particles of claim 19, having an exatecan loading between 1% wt/wt and 25% wt/wt, as measured by high-performance liquid chromatography.

26. The particles of claim 19, wherein the exatecan is in its free base form.

27. The particles of claim 19, wherein:
(i) the particles have a higher proportion of the exatecan encapsulated within the particles than on the surface of the particles, or
(ii) the exatecan is encapsulated within the particles and is not on the surface of the particles.

28. The particles of claim 19, having an average diameter between 1 nm and 1 mm, between 10 nm and 500 nm, between 50 nm and 250 nm, between 50 nm and 200 nm, or between 50 nm and 150 nm, as measured by dynamic light scattering.

29. A method of treating or reducing cancers in a patient, the method comprising:
injecting into a tumor or tissue adjacent to or abutting the tumor the injectable formulation of claim 1, wherein the exatecan or the pharmaceutically acceptable salt thereof is in an effective amount to inhibit tumor growth.

30. The method of claim 29, wherein the tumor is a cutaneous cancer.

31. The method of claim 29, wherein the tumor is a basal cell carcinoma, melanoma, a squamous cell carcinoma, cutaneous T-cell lymphoma, Merkel cell carcinoma, sebaceous carcinoma, Kaposi sarcoma, or dermatofibrosarcoma protuberans.

32. The method of claim 29, comprising administering multiple doses of the formulation.

33. The method of claim 32, comprises injecting a first dose and waiting at least four days before injecting a second dose.

34. The method of claim 29, wherein the formulation is injected via microneedle delivery or needle injection.

35. A method of treating a proliferative disorder or disease, the method comprising administering a formulation comprising the particles of claim 19 to a subject in need thereof, optionally wherein the proliferative disorder or disease is peritoneal carcinomatosis or a cancer, such as skin cancer, esophageal cancer, bladder cancer, ovarian cancer.

36.-38. (canceled)

39. A method of making the particles of claim 19, the method comprising:
(i) adding a base to a solution comprising exatecan.

40.-44. (canceled)
